(19) United States
(12) Patent Application Publication
(10) Pub. No.: US 2020/0270310 A1
(43) Pub. Date: Aug. 27, 2020

(54) MAFG AS A POTENTIAL THERAPEUTIC TARGET TO RESTORE CHEMOSENSITIVITY IN PLATINUM-RESISTANT CANCER CELLS

(71) Applicants: FUNDACÓIN PARA LA INVESTIGACIÓN BIOMÉDICA DEL HOSPITAL UNIVERSITARIO LA PAZ (F1BHULP), Madrid (ES); FUNDACÓIN PARA LA INVESTIGACIÓN BIOMÉDICA DEL HOSPITAL UNIVERSITARIO RAMÓN Y CAJAL (FIBIOHRC), Madrid (ES); FUNDACÓIN PARA LA INVESTIGACIÓN BIOMÉDICA DEL HOSPITAL UNIVERSITARIO LA PRINCESA, Madrid (ES); UNIVERSIDAD AUTÓNOMA DE MADRID, Madrid (ES)

(72) Inventors: Inmaculada IBÁÑEZ DE CÁCERES, Madrid (ES); Javier DE CASTRO CARPEÑO, Madrid (ES); Olga VERA PUENTE, Madrid (ES); Olga PERNÍA ARIAS, Madrid (ES); Carlos RODRÍGUEZ ANTOLÍN, Madrid (ES); Victor Manuel GONZÁLEZ MUÑOZ, Madrid (ES); María Elena MARTÍN PALMA, Madrid (ES); Ana María SALGADO FIGUEROA, Madrid (ES); Rocío ROSAS ALONSO, Madrid (ES); Silvia SACRISTÁN LÓPEZ, Madrid (ES); Rafael LEÓN MARTÍNEZ, Madrid (ES); Patrycja MICHALSKA DZIAMA, Madrid (ES)

(21) Appl. No.: 16/647,368

(22) PCT Filed: Jul. 4, 2018
(86) PCT No.: PCT/EP2018/068156

§ 371 Date: Mar. 13, 2020

(30) Foreign Application Priority Data
Sep. 15, 2017 (EP) 17382610.8

(51) Int. Cl. C07K 7/08; C12Q 1/6804

(52) U.S. Cl. CPC C07K 7/08; C12Q 1/6804; C07K 2317/34; A61K 31/282; C07K 2317/55; C07K 2317/54; C07K 2317/56

(57) ABSTRACT

The present invention relates to the fields of medicine and cancer treatment. The invention more specifically relates to the use of a compound capable of decreasing or inhibiting, in vitro or ex vivo, the expression and/or activity of MAFG and thus increasing ROS-production and chemotherapy sensitivity, specifically in cancer cells resistant to platinum-based therapy. The present disclosure further relates to uses of such compounds, in particular to prepare a pharmaceutical composition to allow or improve the efficiency of a therapy of cancer in a subject in need thereof. The compound of the invention can indeed be advantageously used, in combination with at least one chemotherapeutic dmg, for treating cancer, for preventing cancer metastasis, increasing overall survival and/or for preventing cancer recurrence in a subject. The invention also discloses methods for preventing or treating cancer, cancer metastasis and/or cancer recurrence in a subject, as well as kits suitable for preparing a composition according to the present invention and/or for implementing the herein described methods.

### FIELD OF THE INVENTION

The present invention relates to the fields of medicine and cancer treatment. The invention more specifically relates to the use of a compound capable of decreasing or inhibiting, in vitro or ex vivo, the expression and/or activity of MAFG (musculoaponeurotic fibrosarcoma oncogene family, protein G) and thus increasing ROS-production and chemotherapy sensitivity. The present disclosure further relates to uses of such compounds, in particular to prepare a pharmaceutical composition to allow or improve the efficiency of a therapy of cancer in a subject in need thereof. The compound of the invention can indeed be advantageously used, in combination with at least one chemotherapeutic drug, for treating cancer, for preventing cancer metastasis, increasing overall survival and/or for preventing cancer recurrence in a subject. The invention also discloses methods for preventing or treating cancer, cancer metastasis and/or cancer recurrence in a subject, as well as kits suitable for preparing a composition according to the present invention and/or for implementing the herein described methods.

### BACKGROUND OF THE INVENTION

Cisplatin (CDDP) is the current and most widely used chemotherapeutic agent in several solid malignancies such as lung and ovarian cancers. It is a platinum compound that binds to and crosslinks DNA, thus inducing apoptosis in cancer cells. It has been reported that cisplatin also generates an increase in oxidative stress by increasing levels of superoxide anion, H₂O₂, and hydroxyl radicals that can lead to cell death, improving cisplatin action. However, despite a reasonable rate of initial response, cisplatin treatment often results in chemoresistance development, leading to therapeutic failure in many tumor types. Many studies and reviews in the last 30 years have centered on gaining insight into the molecular mechanism that account for the cisplatin-resistant phenotype of tumor cells, which could provide crucial information for treatment re-sensitizing. A number of events have been proposed as underlying this phenomenon. One such event consists on the ability of cancer cells to overcome the Reactive Oxygen Species (ROS) generated by cisplatin, thus avoiding apoptosis. The complex cellular response against ROS is mainly mediated through the antioxidant response element (ARE), the nuclear factor erythroid-related factor-2 (Nrf2) and the small-Maf-proteins family. Thus, an overexpression of these elements could be responsible of the acquired resistance to cisplatin.

However, there is still a need to provide a method to overcome the strategic challenge of restoring the sensitivity to platinum-derived drugs, this being still the paradigm treatment for many malignancies.

### BRIEF DESCRIPTION OF THE INVENTION

Inventors now provide active molecules that allow the physician to prevent or control, preferably decrease, cancer cell proliferation by increasing ROS-production. They are advantageously capable of increasing the effectiveness of chemotherapeutic treatments directed against any cancer. Inventors herein demonstrate that these molecules are in addition capable of reducing the risk of metastasis and/or cancer recurrence.

The present invention thus relates to the use of a compound capable of decreasing or inhibiting, in vitro or ex vivo, the expression and/or activity of MAFG and thus increasing ROS-production for decreasing chemotherapy resistance. The present disclosure further relates to additional uses of such compounds, in particular to prepare a pharmaceutical composition to allow or improve the efficiency of a therapy of cancer in a subject in need thereof. Moreover, the compound of the invention can indeed be advantageously used, in combination with at least one chemotherapeutic drug, for treating cancer, for preventing cancer metastasis, increasing overall survival and/or for preventing cancer recurrence in a subject. The invention also discloses methods for preventing or treating cancer, cancer metastasis and/or cancer recurrence in a subject, as well as kits suitable for preparing a composition according to the present invention and/or for implementing the herein described methods.

In a particular embodiment, the compound is an aptamer capable of inhibiting the expression and/or activity of MAFG, more particularly a compound of SEQ ID NO 1 (Aptamer apMAFG6F or 6F), wherein SEQ ID NO1 consists of:

GCGGATGAAGACTGGTGTCGATGCATGTGTAGTGCATTTCGAGCCGTT
CTATTGGTGTGCCCTAAATACGAGCAAC

or a pharmaceutically acceptable salt thereof.

In another particular embodiment, the compound is an miRNA capable of inhibiting the expression of MAFG, more particularly miR-7.

It is particularly noted that other preferred compounds useful in the present invention apart from aptamers, are selected from the list consisting of antibodies as well as any fragments thereof, peptides, oligonucleotides, oligonucleotides mimetics, miRNAs and small molecules.

Also herein described is a compound as described above, for use, in combination with at least one chemotherapeutic drug, preferably in combination with chemotherapy regimens based on platinum, more particularly in combination with chemotherapy regimens based on cisplatin and carboplatin, for treating cancer, for preventing cancer metastasis, increasing overall survival and/or for preventing cancer recurrence in a subject.

Another object of the invention is a composition comprising at least one compound capable of decreasing or inhibiting, in vitro or ex vivo, the expression and/or activity of MAFG and thus increasing ROS-production, and preferably at least one chemotherapeutic drug, preferably at least one chemotherapeutic drug based on platinum, more preferably at least cisplatin and carboplatin, to be used simultaneously, separately or sequentially, typically for use for treating cancer, for preventing cancer metastasis, increasing overall survival and/or for preventing cancer recurrence in a subject.

Also herein described is a kit comprising at least one compound capable of decreasing or inhibiting, in vitro or ex vivo, the expression and/or activity of MAFG and thus increasing ROS-production, and at least one chemotherapeutic drug, preferably at least one chemotherapeutic drug based on platinum, more preferably at least cisplatin and/or carboplatin, in distinct containers, as well as uses of said kit typically to prepare a composition according to the present invention.

### BRIEF DESCRIPTION OF THE FIGURES

FIG. 1. Assessment of the methylation levels of miR-7 measured by qMSP and expression levels of miR-7 and MAFG measured by qRT-PCR in fresh samples from a cohort of 22 patients with NSCLC. (A) Left, quantitative methylation percentage of miR-7; middle, miR-7 expression levels; right, MAFG expression levels. (B and C) Correlation between the methylation percentage of miR-7 and expression levels of miR-7 (B) and MAFG (C) in 22 patients with NSCLC. For all the analysis, data represents the percentage of methylation according to previous reports (Eads et al., 2000) and expression levels in 2-ACt.

FIG. 2. Viability assays and Reactive Oxygenated Species detection after CDDP treatment in H23 cell lines. (A) Viability to CDDP in H23S (left) and H23R (right) at 24, 48 and 72 hours and 6 concentrations of CDDP measured by MTT assay. (B) Increment of ROS production in H23S (left) and H23R (right) after 24, 48 and 72 hours of CDDP treatment. $: 72h (vs Basal).

FIG. 3. Viability assays and Reactive Oxygenated Species detection after CDDP treatment in A2780 cell lines. (A) Viability to CDDP in A2780S (left) and A2780R (right) at 24, 48 and 72 hours and 6 concentrations of CDDP measured by MTT assay. (B) Increment of ROS production in A2780S (left) and A2780R (right) after 24, 48 and 72 hours of CDDP treatment. $: 72h (vs Basal).

FIG. 4. Analysis of the affinity of the aptamers against MAFG. (A) Quantification of aptamer-MAFG complexes by qPCR comparing absence (dot pattern) or presence (filled pattern) of MAFG protein. Figure shows the differences in Threshold Cycle (Ct) between the library RND40 and the selection rounds 3 (SEL3MAFG) and 6 (SEL6MAFG). Bars represent the mean±SEM of 2-3 experiments (B) Specificity binding assay of aptamers to MAFG performed by ELONA. MAFG-myc was plated at 50ng/well in 96-well plates followed by incubation with 5′ digoxigenin-labeled aptamers from RND40, SEL3MAFG and SEL6MAFG. The figure represents the mean±SEM of 3 independent experiments. p<0.01. (C) Aptacytochemistry in HEK-293T cells transiently transfected with MAFG-myc. Cells overexpressing MAFG were incubated with 5′ Alexafluor 488-conjugated aptamers from RND40, SEL3MAFG or SEL6MAFG (upper panel) or Myc antibody (lower panel). Confocal microscopy images corresponding to the staining of nuclei with Hoechst (blue), antibodies (red), and aptamers (green) are shown. Arrowhead indicates positive cells. Bar=13 μm. (D) Study of individual aptamers affinity performed by ELONA. Recombinant MAFG was plated at 50 ng/well in 96-well plates followed by incubation with 5′ digoxigenin-labeled individual aptamers apMAGF3F, apMAFG6F and apMAFG11F. The figure represents the mean±SD of 2 independent experiments. (E) Secondary structures of aptamers apMAGF3F, apMAFG6F and apMAFG11F obtained by bioinformatics sequence analysis using the mFold software taking into account the lower free energy (AG).

FIG. 5. Viability curves to increasing doses of aptamers in H23S/R and HEK293T cells. Each experimental group was exposed for 72 h to 6 different aptamers concentrations, and data were normalized to each untreated control, set to 100%. The data represent the mean±SD of at least 3 independent experiments performed in quadruplicate at each concentration for each cell line analyzed.

FIG. 6. Viability curves combining the best dose of aptamers (25nM) and increasing doses of CDDP. Each experimental group was exposed for 72 h to 6 different CDDP concentrations after aptamers transfection; data were normalized to each untreated control, set to 100% and represent the mean±SD of at least 3 independent experiments performed in quadruplicate at each drug concentration for each cell line analyzed 25nM of aptamers and increasing doses of CDDP in H23S/R cells.

FIG. 7: Viability and ROS production assay in H23R cells after apMAFG6F transfection. Each experimental group was exposed for 72 h to 6 different CDDP concentrations, and data were normalized to each untreated control, set to 100%. The data represent the mean±SD of at least 2 independent experiments performed in duplicate at each drug concentration for each cell line analyzed.

FIG. 8. Analysis of expression of MAFG in 2 in silico cohorts of NSCLC patients. (A) Survival analysis in 981 NSCLC samples from the TOGA. (B) Survival analysis in 1035 NSCLC samples from the TCC. LogRank test was used for comparisons and p<0.05 was considered as a significant change in OS. p values in (B) represent the significant difference between overexpressed and downregulated MAFG.

FIG. 9. Characterization of the interaction of aptamers with MAFG. ELONAS in which the Aptamers bound to digoxigenin (20 nM) were incubated 1 hour at 37° C. with 10 peptides (1 μg) previously attached to the plate overnight. After incubating 1 hour with antidigoxigenin-Peroxidase 1/1000, ABTS was added and at 10 minutes the absorbance at 405 nm was measured.

FIG. 10. Structure of the MAFGDNA complex of mouse (accession number 3A5T). HE show two molecules of MAFG (18-111) forming a homodimer (blue and pink). Amino acids corresponding to peptide M4 have been highlighted.

FIG. 11. Amino acids of MAFG with which apMNK6F binds (see chA and chB). Interaction zones of apMNK6F aligned with peptides M4 and M5

### DETAILED DESCRIPTION OF THE INVENTION

Interestingly, we have discovered that CDDP-resistant cells have an increased expression of MAFG, which leads us believe that its involvement in the appearance of CDDP-resistant phenotypes resides in the protection MAFG confers against free radicals generated in the cell after the administration of this drug. Moreover, we have further discovered that the overexpression of MAFG is a result of the downregulation of microRNA-7 by DNA methylation of its regulatory region. For Non-Small Cell Lung Cancer (NSCLC), differential expressions have been reported for various miRNAs, such as miR-138, in the A549 CDDP-resistant subtype cell line compared with the parental A549 and how the enforced expression of miR-138 increased the response to CDDP, or the miR-200c in NSCLC cell lines. However, there is still a need to provide a method to overcome the strategic challenge of restoring the sensitivity to platinum-derived drugs, this being still the paradigm treatment for many malignancies.

In the present invention, we overcome this problem by providing new insight in the role of MAFG in CDDP-resistance development in cancer cell lines and its possible role as a therapeutic target by using compounds, such as aptamers, that show a high affinity to MAFG.

The importance of knowing what genes are involved in the carcinogenic process has increased enormously in recent years. However, a gene's or miRNA function cannot be considered uniquely responsible for the development, progression and resistance to chemotherapy drugs for cancer treatment. The study of epigenetic, transcriptional and posttranscriptional regulatory mechanisms increases our knowledge of the genes and miRNAs involved in these processes. Current treatments can thereby be improved and new ones developed based on the genetic and epigenetic profiles of different cancer patients, thus leading to personalized therapies. The search for new biomarkers for survival, prognosis and drug-resistance in cancer is a wide open field for exploration and investigation. This invention seeks to contribute to the understanding of drug-resistance and the identification of new biomarkers that contribute to this understanding.

To deepen our understanding of the role of miRNAs methylation with tumor development or clinical outcomes, we measured the methylation percentage, the expression levels of miR-7 and expression of MAFG in a cohort of NSCLC patients. We found similar methylation levels in tumor samples from NSCLC patients as well as in non-tumor lung pathologies and no significance was found in terms of therapy response or overall survival in this tumor type regarding miR-7 methylation. We first observed higher dispersion of methylation and expression of both candidates (MIR-7 AND MAFG) in the tumor than in the adjacent-tumor and control samples, suggesting that different molecular changes of miR-7 and MAFG could be mediating the carcinogenic process. We have not specifically observed clear differences either at methylation or expression of any candidate in NSCLC T vs ATT samples, which indicates that the samples adjacent to the tumor have already acquire molecular changes belonging to the tumor. Moreover, the methylation analysis of the ATT compared to the 5 normal saliva samples indicates that the methylation of this miRNA is an event that occurs in the early stages of the disease. This could be a secondary effect of smoking as there is an intense cross-talk between the epigenetics and the environment; in fact, the tobacco smoke and other environmental hazards deregulate the epigenome profile which is closely related to the development of lung cancer. The importance of these data lies in the fact that they were derived from fresh tissues belonging to NSCLC while most of the data in the literature report the expression of miRNAs and candidate genes performed in culture cell lines. Interestingly, we also found a clear relationship between methylation levels and expression of miR-7 and MAFG, which confirm our in vitro results and suggest a relationship with MAFG in NSCLC.

In view of these results, our basic approach was focused to directly link the development of platinum resistance in vitro and the overexpression of MAFG through the decrease of ROS production, in our cellular model. In line with this, the results obtained with the ROS production assay in our cancer cell lines, indicate that the resistant subtype has a less increment of ROS after CDDP exposure, probably by its ability to detoxify the oxidative stress produced by the drug, as a consequence of an overexpression of MAFG. We also found a significant relationship between the expression of MAFG and survival in the bigger cohorts analyzed, with a clear tendency towards more OS when MAFG expression is low. Moreover, inhibition of MAFG through high-affinity aptamers can restore in part the sensitivity to CDDP in cancer cells, through the increase of ROS production, and opens a new possibility in restoring the cisplatin sensitivity in lung cancer. In this sense, we herein provide one aptamer (apMAFG6F) and one miRNA (miR-7) with increasing ROS-production potential, which could be used as therapy in other pathologies related to the decrease of ROS.

To the best of our knowledge, this is the first time describing firstly, the possible role of MAFG as a biomarker of poor prognosis in cancer, in particular in NSCLC; second, its potential as a therapeutic target in cancer and other pathologies through the use of compounds capable of inhibiting the expression of this marker, for example by using aptamers or miRNAs; and thirdly, the possible role of MAFG as a biomarker of treatment response to chemotherapy, in particular as a biomarker of treatment response to chemotherapy regimens based on platinum, more particularly as a biomarker of treatment response to chemotherapy regimens based on cisplatin and carboplatin.

Therefore in a first aspect of the invention, the invention relates to the use of a compound or binding molecule (from hereinafter “compound of the invention”) capable of decreasing or inhibiting, in vitro or ex vivo, the expression and/or activity of MAFG in a cancer cell, as for example in a cancer cell line, in comparison to the expression of MAFG in a non-cancerous cell or in comparison to a reference value (i.e wherein the reference value can be based on the normal expression value of MAFG in in a non-cancerous cell or in a cancerous cells that does not overexpress the MAFG protein), and thus increasing ROS-production, or a pharmaceutically acceptable salt thereof, to decrease or inhibit chemotherapy resistance, in particular, to decrease or inhibit chemotherapy resistance to chemotherapy regimens based on platinum, more preferably to decrease or inhibit resistance to chemotherapy regimens based on cisplatin and carboplatin or another platinum derived compound that gives raise to the development of secondary oxidative stress as a result of its use.

In order to identify compounds of the invention and starting from the amino acidic sequence of human MAFG (NCBI Reference Sequence: NP_116100.2) of: SEQ ID NO 2: MTTPNKGNKALKVKREPGENGTSLTDEELVTMSVRELNQHLRGLSKEEIVQLKQRRRTLKNRG YAASCRVKRVQKEELEKQKAELQQEVEKLASENASMKLELDALRSKYEALQTFARTVARSPVA PARGPLAAGLGPLVPGKVAATSVITIVKSKTDARS); we designed the following 10 peptides (please refer to table below):

| | | | | |
|---|---|---|---|---|
| M1 | MTTPNKGNKALKVKREPGENG | 21 | 10 | 2269.6 |
| M2 | PGENGTSLTDEELVTMSVREL | 21 | 3.91 | 2277.49 |
| M3 | SVRELNQHLRGLSKEEIVQLK | 21 | 8.33 | 2476.86 |
| M4 | IVQLKQRRRTLKNRGYAASCR | 21 | 11.84 | 2517.98 |
| M5 | AASCRVKRVTQKEELEKQKAE | 21 | 9.11 | 2431.79 |
| M6 | KQKAELQQEVEKLASENASMK | 21 | 6.29 | 2389.91 |
| M7 | NASMKLELDALRSKYEALQTF | 21 | 6.18 | 2428.79 |
| M8 | ALQNFARTVARSPVAPARGPL | 21 | 12.30 | 2192.55 |
| M9 | ARGPLAAGLGPLVPGKVAATS | 21 | 11 | 1903.26 |
| M10 | VAATSVITIVKSKTDARS | 18 | 9.99 | 1847.14 |

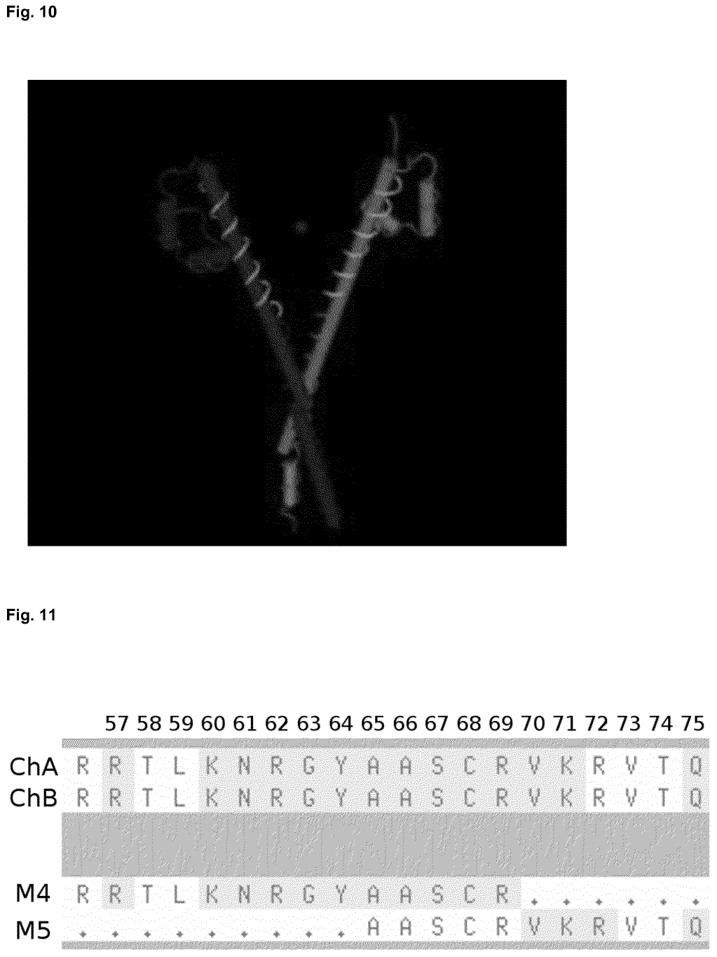

Then, we determined the interaction of aptamers apMNK3F, apMNK6F and apMNK11F (the sequences of each of these aptamers is identified in table 3) with each of the 10 peptides shown above by ELONA, and as illustrated in FIG. 9, the three aptamers recognized peptide M4. Peptide M4, amino acids 49-68 corresponds to the basic MAFG region containing the tyrosine residue (Tyr64), as well as the arginine residue (Arg57) required for binding to the DNA, in addition to a nuclear localization signal. FIG. 10 shows the location of the peptide M4 in the two MAFG chains that form a homodimer of MAFG.

Moreover, we determined the precise amino acids of MAFG with which apMNK6F binds (see chA and chB of FIG. 11). In addition, such interaction zones of apMNK6F where aligned with peptides M4 and M5 (FIG. 11), where it is seen that the interaction occurs mainly with peptide M4, in particular, with an epitope or with an isolated synthetic peptide comprising the amino acid sequence RTLKNRGYAASCR or RTLKNRGYAASCRVKRVTQ. More precisely, the interaction occurs with amino acids 57ARG, 60LYS, 61ASN, 62ARG, 63GLY, 64TYR, 65ALA, 66ALA, 67SER, 68CYS, 69ARG, 70VAL, 71LYS, and 75GLN of the epitope of MAFG comprising amino acids 57 to 75 RTLKNRGYAASCRVKRVTQ (amino acids 57 to 75).

Therefore, by identifying compounds capable of binding to an epitope or to an isolated synthetic peptide comprising the amino acid sequence RTLKNRGYAASCR or RTLKNRGYAASCRVKRVTQ, and capable of decreasing or inhibiting, in vitro or ex vivo, the expression and/or activity of MAFG in a cancer cell, in comparison to the expression and/or activity of MAFG in a non-cancerous cell or in comparison to a reference value, any skilled person shall obtained compounds useful in the first aspect of the invention.

Therefore, in a preferred embodiment of the first aspect, the present invention provides a pharmaceutical composition comprising a binding molecule capable of decreasing or inhibiting, in vitro or ex vivo, the expression and/or activity of MAFG in a cancer cell, in comparison to the expression and/or activity of MAFG in a non-cancerous cell or in comparison to a reference value, for use in a method to decrease or inhibit chemotherapy resistance to chemotherapy regimens based on platinum, wherein said binding molecule binds to an epitope or to an isolated synthetic peptide comprising the amino acid sequence RTLKNRGYAASCR or RTLKNRGYAASCRVKRVTQ, alternatively said binding molecule binds to an epitope or to an isolated synthetic peptide comprising the amino acid sequence IVQLKQRRRTLKNRGYAASCR or IVQLKQRRRTLKNRGYAASCRVKRVTQ.

In another preferred embodiment of the first aspect, the binding molecule is a compound selected from the group consisting of aptamers, antibodies, and antibody fragments selected from the list consisting of Fv, scFv, Fab, F(ab′)2, Fab′, scFv-Fc, diabodies, or any fragment whose half-life has been increased by chemical modification. Preferably, such compound is an aptamer. Aptamers are oligonucleotide or peptide molecules that bind to a specific target molecules. Aptamers are usually created by selecting them from a large random sequence pool, but natural aptamers can also exist in riboswitches. More specifically, aptamers can be classified as: DNA or RNA aptamers and comprise strands of oligonucleotides. Peptide aptamers comprise short variable peptide domains, attached at both ends to a protein scaffold. More preferably, such aptamer is aptamer apMAFG6F.

In yet another preferred embodiment of the first aspect, the binding molecule binds to aminoacids 57ARG, 60LYS, 61ASN, 62ARG, 63GLY, 64TYR, 65ALA, 66ALA, 67SER, 68CYS, 69ARG, 70VAL, 71LYS, and 75GLN of the epitope of MAFG comprising amino acids 57 to 75 RTLKNRGYAASCRVKRVTQ (amino acids 57 to 75). Preferably, said binding molecule is an aptamer, more preferably the aptamer is apMAFG6F of SEQ ID NO 1.

In another preferred embodiment, such compound of the invention is selected from the group consisting of oligonucleotides, mimetics, miRNAs, peptides or small molecular weight (MW) compounds. which bind to MAFG and are capable of decreasing or inhibiting, in vitro or ex vivo, the expression and/or activity of MAFG in a cancer cell, as for example in a cancer cell line, in comparison to the expression of MAFG in a non-cancerous cell or in comparison to a reference value.

As used herein, the term “pharmaceutically acceptable” refers to compositions, compounds, salts and the like that are, within the scope of sound medical judgment, suitable for contact with the tissues of the subject, or which can be administered to the subject, without excessive toxicity or other complications commensurate with a reasonable benefit/risk ratio. For instance, pharmaceutically acceptable salts encompass sodium, potassium, chloride, ammonium, acetate salts and the like.

The compounds of the invention can be obtained by methods well-known by the skilled artisan.

The invention also relates to the use of at least one compound of the invention or a pharmaceutically acceptable salt thereof for increasing the sensitivity of a cancer to a chemotherapeutic agent, preferably for increasing the sensitivity of a cancer to chemotherapy regimens based on platinum, more preferably for increasing the sensitivity of a cancer to chemotherapy regimens based on cisplatin and carboplatin or another platinum derived compound.

A further object of the invention is the use of at least one compound of the invention or a pharmaceutically acceptable salt thereof for decreasing the resistance of a cancer with respect to a chemotherapeutic agent, preferably for decreasing the resistance of a cancer to chemotherapy regimens based on platinum, more preferably for decreasing the resistance of a cancer to chemotherapy regimens based on cisplatin and carboplatin. Also described is a compound of the invention (or a pharmaceutically acceptable salt thereof), for use, in combination with at least one chemotherapeutic drug, chemotherapy regimens based on platinum, or chemotherapy regimens based on cisplatin and carboplatin, for treating cancer, for preventing cancer metastasis, increasing overall survival and/or for preventing cancer recurrence in a subject.

The term “subject” refers to any subject and typically designates a patient, in particular a subject undergoing a treatment of cancer such as chemotherapy and/or radiotherapy, or a subject at risk, or suspected to be at risk, of developing a cancer. The subject is preferably a mammal, even more preferably a human being, for example a human being suffering of a cancer and resistant to chemotherapy. The subject is typically a cancer patient, and the patient is preferably resistant to chemotherapy. The subject may have been exposed to part of a complete conventional treatment protocol, for example to at least one cycle of the all treatment protocol, for example two cycles of the all treatment protocol.

The cancer may be any kind of cancer or neoplasia. A typical cancer is a cancer resistant to the first-line chemotherapy. The cancer is for example selected from a melanoma (Cretnick et al. 2009), a breast cancer Smith et al. 2014, Jeng ks et al. 2013), a thyroid cancer (Hinterseher al. 2014, Xu H. et al. 2012), a prostate cancer (Kim et al. 2011, Chung et al. 2010), a colon cancer (Wang et al. 2013, Xu M. et al. 2012), a rectal cancer (Qualtrough et al. 2004), an oesophagus cancer (Zhu W. et al. 2011), a gastric cancer Lee S J et al. 2013), an ovarian cancer (Sabo) et al. 2012), a lung cancer (Li et al. 2012, Gialmanidis et al. 2009), a pancreatic cancer (Ma et al. 2014, Nakamura et al. 2012), a glioma (Yu et al. 2014), an adrenocortical carcinoma (Mus-Veteau et al., unpublished data), pediatric solid malignant tumors such as a neuroblastoma, a rhabdomyosarcoma, a nephroblastoma or a hepatoblastoma (Oue et al. 2010), but also non solid cancers, for example a leukaemia such as a lymphoid leukemia or a myeloid leukemia (Cea et al. 2013), a multiple myeloma (Blotta et al. 2012), and a sarcoma such as an osteosarcoma (Lo et al. 2014).

In a particular embodiment of the present invention, the chemotherapeutic agent is a platinum based component. Examples of platinum-based components are CDDP and OXP.

The treatment which can include several chemotherapeutic agents is selected by the cancerologist depending on the specific cancer to be prevented or treated.

In particular embodiments of the invention Cisplatin, OXP or carboplatin is used in combination with at least one compound of the invention.

The present disclosure further relates to use of a compound of the invention as defined above (including anyone of the disclosed embodiments) to prepare a pharmaceutical composition or medicament, said composition being capable of allowing or of improving the efficiency of a therapy of cancer in a subject in need thereof. The compound of the invention can in particular be advantageously used, in combination with at least one chemotherapeutic drug, chemotherapy regimens based on platinum, or chemotherapy regimens based on cisplatin and carboplatin, for treating cancer, for preventing cancer metastasis, increasing overall survival and/or for preventing cancer recurrence in a subject.

A preferred pharmaceutical composition thus comprises, as a combined preparation, at least one drug used in a treatment of a cancer, typically at least one chemotherapeutic drug, chemotherapy regimens based on platinum, or chemotherapy regimens based on cisplatin and carboplatin, as herein described for simultaneous, separate or sequential use with a compound of the invention in the treatment of cancer.

Herein described are also (i) a method for preventing or treating cancer, (ii) a method for increasing the sensitivity of a cancer to a chemotherapeutic agent, chemotherapy regimens based on platinum, or chemotherapy regimens based on cisplatin and carboplatin, and (iii) a method for decreasing the resistance of a cancer with respect to a chemotherapeutic agent, chemotherapy regimens based on platinum, or chemotherapy regimens based on cisplatin and carboplatin, each of said methods comprising administering a subject in need thereof with an effective amount of at least one compound of the invention as defined above including anyone of the disclosed embodiments or a pharmaceutical composition as defined above, preferably together with a chemotherapeutic drug, a chemotherapy regimen based on platinum, or a chemotherapy regimen based on cisplatin and carboplatin, classically used in the prevention or treatment of cancer as herein described (as a combined preparation).

In another particular embodiment, said method further comprises administering an effective amount of another therapeutically active compound for preventing or treating cancer or a cancer treatment side effect.

By “treatment” is meant the curative treatment of cancer. A curative treatment is defined as a treatment that completely treat (cure) or partially treat (induces tumor growth stabilization, retardation or regression) cancer.

As used herein, “a therapeutically effective amount or dose” refers to an amount of the compound of the invention which prevents, removes, slows down the cancer or reduces or delays one or several symptoms or disorders caused by or associated with said disease in the subject, preferably a human being. The effective amount, and more generally the dosage regimen, of the compound of the invention and pharmaceutical compositions thereof may be determined and adapted by the one skilled in the art. An effective dose can be determined by the use of conventional techniques and by observing results obtained under analogous circumstances. The therapeutically effective dose of the compound of the invention will vary depending on the disease to be treated or prevented, its gravity, the route of administration, any co-therapy involved, the patient's age, weight, general medical condition, medical history, etc.

In a particular aspect, the compounds of the invention can be administered to the subject by parenteral route, oral route or intraveinous (IV) injection. The compound or the nanoparticle of the invention may be administered to the subject daily (for example 1, 2, 3, 4, 5, 6 or 7 times a day) during several consecutive days, for example during 2 to 10 consecutive days, preferably from 3 to 6 consecutive days. Said treatment may be repeated during 1, 2, 3, 4, 5, 6 or 7 weeks, or every two or three weeks or every one, two or three months. Alternatively, several treatment cycles can be performed, optionally with a break period between two treatment cycles, for instance of 1, 2, 3, 4 or 5 weeks.

The administration route can be oral or parenteral, typically rectal, sublingual, intranasal, intra- peritoneal (IP), intra-veinous (IV), intra-arterial (IA), intra-muscular (IM), intra-cerebellar, intrathecal, intratumoral and/or intradermal. The pharmaceutical composition is adapted for one or several of the above-mentioned routes. The pharmaceutical composition is preferably administered by injection or by intravenous infusion of suitable sterile solutions, or in the form of liquid or solid doses via the alimentary canal.

The pharmaceutical composition can be formulated as solutions in pharmaceutically compatible solvents or as gels, oils, emulsions, suspensions, or dispersions in suitable pharmaceutical solvents or vehicles, or as pills, tablets, capsules, powders, suppositories, etc. that contain solid vehicles in a way known in the art, possibly through dosage forms or devices providing sustained and/or delayed release. For this type of formulation, an agent such as cellulose, lipids, carbonates or starches are used advantageously.

Agents or vehicles that can be used in the formulations (liquid and/or injectable and/or solid) are excipients or inert vehicles, i.e. pharmaceutically inactive and non-toxic vehicles.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations suitable for parenteral administration conveniently comprise a sterile oily or aqueous preparation of the active ingredient which is preferably isotonic with the blood of the recipient. Every such formulation can also contain other pharmaceutically compatible and non-toxic auxiliary agents, such as, e.g. stabilizers, antioxidants, binders, dyes, emulsifiers or flavouring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

Another object of the invention is a kit comprising at least one compound according to the invention and preferably at least one chemotherapeutic drug, a chemotherapy regimen based on platinum, or a chemotherapy regimens based on cisplatin and carboplatin, in distinct containers. The kit can further comprise instructions for preparing a composition according to the invention, for carrying out anyone of the herein described method, for example for preventing or treating cancer, for preventing or treating cancer metastasis and/or for preventing or treating cancer recurrence in a subject.

In a particular embodiment, the present invention relates to the use of a kit according to the invention to prepare a composition as herein described.

In another particular embodiment, the kit is suitable for implementing anyone of the herein described method, in particular a method for treating cancer, for preventing cancer metastasis, increasing overall survival and/or for preventing cancer recurrence in a subject.

Another aspect of the invention is based on examples 2 and 3 of the present specification. In this sense, as exemplified therein, it is again noted that epigenetic alterations by DNA methylation can reduce the expression of a number of miRNAs, altering the therapeutic response in tumor cells and contributing to the onset of more aggressive phenotypes. In order to deepen our understanding of this aspect, identifying new miRNA-targets of promoter hypermethylation involved in the response to cisplatin, by using an experimental model of paired sensitive and CDDP-resistant tumor cell lines. We established the ovarian cancer cells A2780R and OVCAR3R, and the lung cancer cell lines H460R and H23R, with a CDDP-RI. Then, we combined an epigenetic reactivation therapy with a global transcriptomic-based strategy. The epigenetic therapy induced an expected reactivation percentage of 12% (87 of the 723 miRNAs). The differential miRNA expression profile from sensitive, CDDP-resistant and resistant cells under epigenetic reactivation treatment was correlated with the expression of those genes from the same experimental groups that showed complementary sequences. This screening, included an ontological study of routes and processes related to tumor biology for all candidate target genes. We identified a set of 7 miRNAs containing a surrounding CGI that were complementary to target genes involved in cell growth, proliferation, cell migration, drug efflux, angiogenesis or apoptosis inhibition such MAFG, ELK-1, RAB6B,CAMK2G, MAPKAP1, ABCA1, ABL1 or STAT3. All these processes might influence the acquisition of drug-resistance in the CDDP treated cells through the potential miRNA silencing.

Changes in expression were validated for all 7 candidates, but not in all the expected paired cell lines, indicating that qRT-PCR is a valuable and necessary validation method more restrictive than microarray, that still keeps providing a powerful tool to study the involvement of a large number of miRNAs simultaneously. The expression changes were more significant after unmasking treatment, probably because the pharmacologic combination exerts a synergistic and specific influence in mRNA and miRNA global re-expression, as described in different tumor types. This effect can be stronger than the silencing observed as a secondary effect of CDDP on DNA methylation.

The expression of miR-7, miR-132, miR-335 and miR-148a was validated in at least two paired cell lines. Epigenetic validation of those candidates revealed that only one miRNA from our panel, miR-7, had specific methylation in CDDP-resistance. miR-148a and miR-132 expression might be regulated by an upstream epigenetic mechanism or transcription factor reactivated by demethylation. miR-335 epigenetic reactivation has been reported in breast cancer cells, confirming the relevance of our approach to identifying miRNAs under epigenetic regulation, although the response after platinum treatment was not studied. We found specific methylation in both, H460S/R cells, but not in the controls and additional cell lines analyzed, suggesting that the downregulation in the resistant phenotypes is probably independent of the methylation profile. We cannot conclude that the downregulation of miR-335 is affecting the response to platinum, but the sensitivity to the drug seems not to be mediated by DNA methylation.

We focused on the epigenetic regulation of miR-7 at the upstream CpG island analyzed, as the one encompassing miR-7 showed constitutive methylation, suggesting the absence of a regulatory role, as reported for other potential regulatory CGIs. The same methylation profile was found in ovarian, lung, colon and pancreatic cisplatin-resistant cell lines. These data suggest a potential epigenetic regulation of miR-7 at DNA methylation level for this second CGI, a relatively common event in various tumor types, which can present intrinsic resistance to CDDP by epigenetic regulation. We therefore tested the specificity of aberrant miR-7 hypermethylation as a potential epigenetic biomarker to detect the response to chemotherapy on 167 ovarian cancer patients, all of them treated with platinum-based therapy. A clinical follow-up of 83 of those patients showed that those considered platinum-sensitive, harboring an unmethylated miR-7 had a better progression free survival rates than those patients with a methylated marker. These differences were not observed in platinum-resistant patients, probably because in these patients the recurrence develop in short-time periods and in a small number of cases. We confirmed the same tendency in an additional smaller cohort of 55 patients. Furthermore, our analysis indicated that those patients carrying an unmethylated marker tended to have less aggressive tumors, with three times more overall survival after platinum treatment than those who carried the methylated DNA. In addition, the methylation percentage increased in tumor grades III/IV and when analyzing high-serous samples and Platinum-refractory/resistant tumors. Thus, miR-7 methylation plays a role as a clinical tool predicting the aggressive behavior of this malignancy and the poorer response to platinum-based treatment.

We sought then to confirm the role of miR-7 in the response to CDDP, in order to explore the potential therapeutic effect of miR-7 overexpression. However, the ectopically overexpression of miRNA-7 in resistant cells did not change their sensitivity to CDDP, although it induced an increase in cell mortality; probably, due to the multifactorial effect that overexpression of miRNAs may cause on the cellular processes by regulating a high number of potential candidates genes. Its expression has been also linked with sensitization to paclitaxel, although its regulation in this process has not being defined. miR-7 might be involved in these processes through the regulation of its target genes, whose overexpression has been found in our experimental approach. Using a transcriptomic profile together with the in silico assembling of sequences, we identified a group of genes candidate to be targets of miR-7 that could provide cells with oncogenic capabilities. Further analysis of molecular pathways and cellular functions, led us to the selection of MAFG, ELK-1, ABCA1 and MAPKAPI genes. Validation by alternative techniques and overexpression of miR-7 in the resistant cell lines, revealed that MAFG, ABCA1 and ELK-1 recovered their levels of expression after epigenetic treatment and overexpression of miR-7, thus indicating a possible regulation of these genes by the methylation of this miRNA. However, our functional studies performed with luciferase vectors carrying a mutation in the conserved miR-7 binding site, revealed that only MAFG was a direct candidate target gene under miR-7 regulation. Moreover, the silencing of miR-7 expression resulted in increased levels of MAFG and its overexpression is able to strongly increase the resistance to CDDP in sensitive cells.

Despite the fact that sMafs family, to which MAFG belongs, have been associated with cellular response, little is known about their involvement in human diseases. A number of studies have however linked these proteins with cancer, such as the study by Schembri et al. on MAFG regulation by miR-218 as an indicator of smoking-induced disease processes in the lungs and the study by Yang et al. on the relationship between increased MAFG and growth in colon cancer cell lines through the insulin-like growth factor-I actions. Taken together, our experimental results strongly support the direct regulation of MAFG through miR-7 and their involvement in the development of CDDP resistance in human tumor cells.

In the present invention, we introduce the epigenetic regulation of miR-7 as a mechanism involved in platinum-resistance in cancer cell lines directly regulating the action of MAFG, which is overexpressed in resistant phenotypes. To the best of our knowledge, this is the first report linking the regulation of MAFG by miRNA-7 and its role in chemotherapy response to CDDP. Moreover, miR-7 methylation arises as a potential predictive biomarker for the identification of ovarian cancer patients that may present worst response to platinum-derived treatment in terms of Overall Survival (hereinafter called, OS) and Progression-Free Survival (hereinafter called, PFS). Furthermore, this data captures the interest of researchers due to the possible role MAFG plays as a novel therapeutic target for platinum resistant tumors.

Therefore, a further aspect of the invention refers to the use of MAFG as biomarker of poor prognosis in cancer, preferably in terms of OS and PFS, also preferably in NSCLC and ovarian cancer. Please note that such further aspect of the invention, has been validated as illustrated in example 3.

Furthermore, another aspect of the invention, refers to the role of MAFG as a biomarker of treatment response to chemotherapy, in particular as a biomarker of treatment response to chemotherapy regimens based on platinum, more particularly as a biomarker of treatment response to chemotherapy regimens based on cisplatin and carboplatin.

The disclosure thus provides methods for predicting the likelihood that a human patient with a cancer will exhibit a beneficial response to chemotherapy, in particular as a biomarker of treatment response to chemotherapy regimens based on platinum, more particularly as a biomarker of treatment response to chemotherapy regimens based on cisplatin and carboplatin, based on expression levels of at least MAFG in a biological sample from the patient, preferably from a human sample obtained from a tumor in the patient. Specifically, the method entails measuring an expression level of at least the MAFG gene, or its expression product. The expression level is normalized, and the normalized expression level is used to determine or predict likelihood of beneficial response, wherein increased normalized expression levels of MAFG are positively correlated with the likelihood that the patient will not exhibit a beneficial response to chemotherapy, in particular to treatment response to chemotherapy regimens based on platinum, more particularly to treatment response to chemotherapy regimens based on cisplatin and carboplatin. A report is generated based on the determined likelihood of response.

A normalized expression level(s), generated as discussed above, is used to determine or predict likelihood of beneficial response, A likelihood score (e.g., a score predicting likelihood of beneficial response to chemotherapy treatment) can be calculated based on the normalized expression level(s). A score may be calculated using weighted values based on a normalized expression level of a response indicator gene.

In addition, the disclosure provides arrays for carrying out the methods disclose herein, or for analyzing whether a mathematical combination of the normalized expression levels of any combination of the response indicator genes is more indicative of a likelihood that a patient will respond to treatment.

Determining the expression level of MAFG may be accomplished by, for example, a method of gene expression profiling. The method of gene expression profiling may be, for example, a PCR-based method. The expression level of said genes can be determined, for example, by RT-PCR (reverse transcriptase PCR), quantitative RT-PCR (qRT-PCR), or other PCR-based methods, immunohistochemistry, proteomics techniques, an array-based method, or any other methods known in the art or their combination.

The expression levels of the genes may be normalized relative to the expression levels of one or more reference genes, or their expression products.

The tumor sample may be, for example, a tissue sample containing cancer cells, or portion(s) of cancer cells, where the tissue can be fixed, paraffin-embedded or fresh or frozen tissue. For example, the tissue may be from a biopsy (fine needle, core or other types of biopsy) or obtained by fine needle aspiration, or by obtaining body fluid containing a cancer cell, e.g. urine, blood, etc.

For all aspects of the methods of the present disclosure, it is contemplated that for every increment of an increase in the level of MAFG, the patient is identified to show an incremental decreased in clinical outcome.

The determination of expression levels may occur more than one time in the practice of the methods disclosed herein.

The methods may further include the step of creating a report based on the determined or predicted likelihood of beneficial response. In another aspect the present disclosure provides reports for a patient containing a summary of the expression levels of MAFG, in a tumor sample obtained from said patient. In one aspect the report is in electronic form.

In one embodiment, the expression levels of MAFG are detected by using an antibody specific for MAFG. In another, the expression levels of MAFG are detected by using a small molecule, in yet another, the expression levels of MAFG are detected by using an aptamer, in particular any of the aptamers illustrated in example 1 or any of the compounds of the invention identified in the first aspect of the invention.

Lastly, as regards the use of MAFG as biomarker of poor prognosis in cancer, preferably in terms of OS and PFS, also preferably in NSCLC and ovarian cancer, the same considerations and tools applicable to the determination of the role of MAFG as a biomarker of treatment response to chemotherapy, are applicable here.

Further aspects and advantages of the present invention will be disclosed in the following experimental section which shall be considered as illustrative only.

### EXAMPLES

### Example 1

### MAFG is a Potential Therapeutic Target to Restore Chemosensitivity in Platinum-Resistant Cancer Cells by Increasing Reactive Oxygen Species

### Materials and Methods

Cell cultures and treatments

The human cancer cells H23 and A2780, and the epithelial cells HEK-293T were purchased from ATCC (Manasas, USA) or ECACC (Sigma, Spain) and cultured as recommended. The CDDP-resistant variants H23R and A2780R were established in our laboratory as previously described (14) (Vera et al, 2017), using CDDP (Farma Ferrer, Spain) for cell viability assays.

### Clinical Sample and Data Collection

Fresh frozen tumor (T) and Adjacent-tumor tissues (ATT) paired samples were obtained from 22 NSCLC patients from Hospital la Paz. All patients had both a perioperative PET-CT scan showing localized disease and a pathological confirmation of stages after undergone a complete resection for a histologically confirmed early NSCLC. Follow-up was conducted according to the criteria of the medical oncology divisions from each institution. In addition, 5 saliva samples from healthy donors were used as controls. All samples were collected after the approval of the appropriate Human Research Ethics Committee at the contributing center, including an informed consent within the context of research. Clinical, pathological and therapeutic data were recorded by an independent observer, and a blind statistical analysis was performed on the data.

### RNA Isolation and Quantitative RT-PCR

Total RNA from surgical samples was isolated, reverse transcribed and quantitative RT-PCR analysis were performed as previously described (14, 15). Samples were analyzed in triplicate using the HT7900Real-Time PCR system (Applied Biosystems, USA), and relative gene expression quantification was calculated according to the comparative threshold cycle method (2^{-ΔΔCt}) using GADPH as an endogenous control gene. Primers and probes for expression analysis were purchased from Applied Biosystems (MAFG: Hs 01034678_g1; GADPH: Hs03929097_g1).

### DNA Extraction, Bisulfite Modification, Methylation-Specific PCR and Quantitative Methylation Specific PCR

DNA from a total of 44 surgical samples from patients with NSCLC was isolated and bisulfite modified and was used to analyze miRNA-7 methylation status. For quantitative Methylation-Specific PCR (qMSP), we used the primer/probe set to detect levels of either methylation (F: 5′-GGGTGGGGTTTTTTAAGAATC-3′; R: 5′-ACATTCTCCTCCTTCGATCG-3′; Probe: 5′-FAM-ACCCCTCTTCGTTCTCGAT-3′) or unmethylation (F: 5′-GGGGTGGGGTTTTTTAAGAATT-3′; R: 5′-ATAACATTCTCCTCCTTCAATCA-3′; Probe: 5′-VIC-ACCCCTCTTCATTCTCAAT-3′). All assays were performed by duplicate using the QuantiTect Multiplex PCR Kit (Qiagen, USA) and the HT7900 Applied Biosystems. The percentage of methylation of each sample was calculated according to previously published reports.

### ROS Measurement

H23/A2780 cells were cultured in 96-well black plates at density of 10,000 cells/well. Cells were treated with 6 different doses of CDDP for 24, 48 and 72 h in RPMI containing 10% FBS. Then treatments were removed and cells were loaded with the fluorescent probe 2′,7′-dichlorodihydrofluorescein diacetate (H2DCFDA) (10 μM) for 45 minutes in RPMI medium (FBS free). Cells were washed twice with RPMI (10% FBS), and fluorescence was recorded in a Fluostar Optima, each hour, over 4 hours period. At the end of the experiment, solutions were replaced for fresh media containing MIT (0.5 mg/mI concentration) in order to determinate viability. ROS production was calculated dividing the mean H2DCFDA fluorescence by the mean viability. Data were normalized with respect to basal conditions that were considered as 100%. All experiments included cells treated with RPMI alone (Basal), and one well without cells as fluorescence basal.

### Aptamers Selection for MAFG

Selection of DNA aptamers for recombinant MAFG was performed as described previously (Ramos et al.; Molecular Therapy-Nucleic Acids (2016) 5, e275). Briefly, synthetic random ssDNA (IBA Life Sciences, Germany), containing a central randomized region of 40 nucleotides flanked by two conserved 18-nucleotides regions in each end (RND40, 5′-GCGGATGAAGACTGGTCT-40N-GTTGCTCGTATTT AGGGC-3′), was denatured at 95° C. for 10 minutes and then cooled on ice for 10 minutes. For the initial SELEX round, 340 pmol of RND40 were mixed with 75 pmol of MAFG in 200 pl of selection buffer (20 mmol/l Tris-HCl pH 7.4, 1 mmol/l MgCl2, 150 mmol/l NaCl, and 5 mmol/l KCl) and incubated at 37° C. for 1 hour. The aptamer-MAFG complexes were purified by adding 20 pl of Ni-NTA superflow (Qiagen, Spain) for 1 hour at 4° C. After washing three times with 1 ml of selection buffer, the aptamer-MAFG complexes were suspended in 20 μl of distilled H2 O and amplified by PCR using the primers named F3 (5′ GCGGATGAAGACTG GTGT 3′) and R3 (5′ GTTGCTCGTATTTAGGGC 3′) (IBA Life Sciences, Germany) under the conditions of 0.8 μmol/l/primer, 200 μmol/l dNTPs, 2 mmol/l MgCl2 , and 2U Taq polymerase (Biotools, Spain) in a final volume of 50 μl for 15 cycles (95° C. for 30 seconds, 56° C. for 30 seconds, and 72° C. for 30 seconds). In the next rounds of selection, 25 μg (1 nmol) of previously selected population were denatured at 90° C. for 10 minutes and then cooled on ice for 10 minutes and used as above. Contraselection against Ni-NTA resin was performed after rounds 3 and 6.

### Aptamers Transfection and Viability Assays

To identify the more effective and less harmful dose of aptamers HEK and H23S/R cells were seeded into 96-well plates then transfected with 50, 125, 250, 750 and 1000 nM of MAFG-aptamers or treated with PBS-Mg using Jet Pei polyplus transfection reagent (PolyPlus Transfection, USA). Once identified the best dose of aptamers, cells were transfected with aptamers and treated with increased doses of CDDP to assay viability and ROS production as mentioned above. In parallel, for all the experiments, cells were seeded in 24-well plates for aptamers extraction and confirmation of aptamers transfection.

### Analysis of Aptamer-Transfection Efficiency by Real-Time PCR

Aptamers were isolated by incubating the cells with ultrapure H2O at 90° for 10 minutes, and centrifugation at 12000 g for 10 minutes. Quantitative analysis was performed by qPCR using PowerUp™ SYBR® Green Master Mix (Thermo Fisher Scientific, USA) and F3 and R3 oligonucleotides following the manufacturer's instructions in the HT7900 Applied Biosystems (Applied Biosystems, USA). The reaction mixture consisted of 1× using PowerUp™ SYBR® Green Master Mix, 0.2 μmol/l oligonucleotide, and 1 μl of lysis supernatant in a 20 μl/tube final volume.

### Statistical Analysis

The data were compared with the chi-squared test or Fisher's exact test for qualitative variables, and Student's t test or the Wilcoxon-Mann-Whitney test (non-normal distribution) for quantitative variables. For in silico databases, the data were stratified for patients with high or low expression of MAFG according to the mean of the gene expression (TOGA cutoff: 2018 Counts Per Million; TCC cutoff: 8.70 of intensity probe). Overall survival was estimated according to the Kaplan-Meier method and compared between groups by means of the Log Rank test. All the p-values were two-sided, and the type I error was set at 5 percent. Statistical analyses were performed using SPSS 20 software.

### Results

miR-7 Methylation is in Part Responsible of MAFG Upregulation in Human NSCLC Primary Tumors

We have discovered that epigenetic regulation of miR-7 is a mechanism involved in platinum-resistance in cancer cell lines directly regulating the action of MAFG, which is overexpressed in resistant phenotypes. Thus, in the present invention we aimed to explore the involvement of miR-7/MAFG axis in NSCLC patients. We first analyze the quantitative methylation levels of miR-7 in a cohort of Fresh frozen-paired tumor samples (T) and adjacent-tumor tissues (ATT) from patients with NSCLC (Table 1). 5 normal saliva samples were used as control for the comparisons. Fresh tissues were also used for RNA isolation to analyze the relative expression levels of miR-7 and MAFG and correlate them with miR-7 methylation levels.

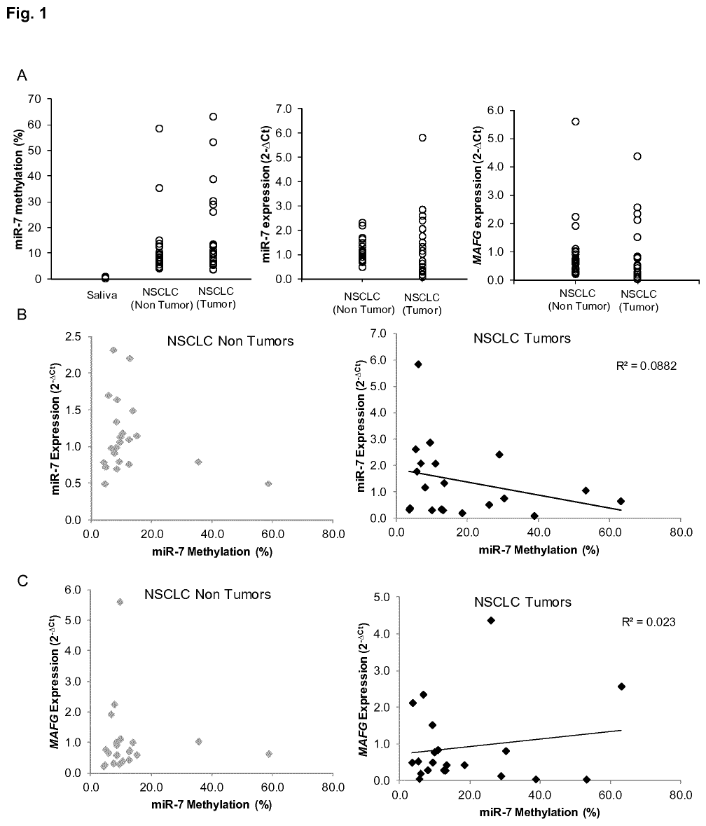

We observed higher variability in the tumor samples than in the adjacent ones, at methylation and expression levels (FIG. 1A). These results also shown an increase in the data dispersion at the T vs the ATT samples and saliva in NSCLC patients that was statistically significant only when comparing T or ATT samples with saliva (p=0.0002) (FIG. 1A and Table 2). Then, we studied the correlation of the miR-7 methylation with MAFG and miRNA expression data, and we observed a negative relation between the methylation percentage and the expression of miR-7 in NSCLC tumor samples (FIG. 1B) and a tendency towards more expression of MAFG in NSCLC when the percentage of miR-7 methylation increases (FIG. 10). Interestingly, the two samples that showed the highest dispersion for MAFG expression presents a miR-7 methylation level over 20%.

TABLE 1
Clinicopathological data and hypermethylation frequence
of patients with NSCLC from Hospital la Paz.

| | | | | | | miR-7methylation | |
|---|---|---|---|---|---|---|---|
| | | Age, | | | | ATT | T |
| Patient | Gender | years | Histology | Stage | Chemotherapy | (%) | (%) |
| 1 | Female | 56 | Adenocarcinoma | IA | No | 58.49 | NA |
| 2 | Male | 69 | Epidermoid | IB | No | 5.63 | 5.35 |
| 3 | Male | 71 | Adenocarcinoma | IB | No | 14.98 | 13.12 |
| 4 | Male | 79 | Adenocarcinoma | NA | No | 12.47 | 26.06 |
| 5 | Male | 74 | Large Cell | IIB | No | 9.20 | 12.69 |
| 6 | Male | 62 | Adenocarcinoma | IIIA | Other | 7.28 | 3.74 |
| 7 | Female | 63 | Epidermoid | IB | CDDP-other | 8.47 | 5.69 |
| 8 | Female | 41 | Adenocarcinoma | IIA | CDDP-other | 8.37 | 28.94 |
| 9 | Male | 86 | Epidermoid | IB | No | 9.41 | 8.09 |
| 10 | Male | 58 | Adenocarcinoma | IA | No | 8.19 | 38.79 |
| 11 | Female | 61 | Adenocarcinoma | IIIA | CBDCA-others | 6.51 | 6.08 |
| 12 | Male | 62 | Epidermoid | IB | CDDP-other | 7.59 | 30.28 |
| 13 | Male | 79 | Epidermoid | IIA | No | 12.44 | NA |
| 14 | Male | 81 | Epidermoid | IIA | No | 12.66 | 10.98 |
| 15 | Female | 66 | Adenocarcinoma | IIIA | CDDP-other | 4.72 | 3.57 |
| 16 | Male | 74 | Adenocarcinoma | IIB | Other | 35.35 | 10.00 |
| 17 | Male | 84 | Epidermoid | IIB | CBDCA-others | 8.24 | 53.19 |
| 18 | Female | 55 | Adenocarcinoma | IB | CDDP-other | 10.29 | 9.42 |
| 19 | Male | 57 | Epidermoid | IIB | CDDP-other | 13.67 | 9.52 |
| 20 | Male | 74 | Epidermoid | IIIB | CBDCA-others | 4.06 | 13.45 |
| 21 | Male | 65 | Adenocarcinoma | IIIA | CDDP-other | 9.55 | 63.07 |
| 22 | Female | 35 | Adenocarcinoma | IIB | CDDP-other | 4.43 | 6.84 |

| | miR-7 expression | | MAFG expression | | | | Overall |
|---|---|---|---|---|---|---|---|
| | ATT | T | ATT | T | Last | | Survival, |
| Patient | (2-ΔCt) | (2-ΔCt) | (2-ΔCt) | (2-ΔCt) | Contact | Status | months |
| 1 | 0.50 | 0.19 | 0.62 | 0.43 | May 18, 2015 | Alive | 37 |
| 2 | 1.71 | 2.61 | 0.65 | 0.53 | Oct. 15, 2014 | Exitus | 34 |
| 3 | 1.16 | 0.31 | 0.59 | 0.28 | Aug. 1, 2011 | Exitus | 1 |
| 4 | 1.11 | 0.51 | 0.43 | 4.37 | Mar. 21, 2013 | Exitus | 20 |
| 5 | 0.80 | 0.34 | 0.28 | 0.29 | Nov. 25, 2011 | Exitus | 3 |
| 6 | 2.33 | 0.38 | 0.31 | 2.12 | Nov. 10, 2011 | Exitus | 8 |
| 7 | 1.65 | 1.77 | 0.58 | 0.05 | Apr. 29, 2011 | Exitus | 7 |
| 8 | 0.70 | 2.42 | 0.91 | 0.13 | Sep. 17, 2016 | Alive | 56 |
| 9 | 1.07 | 1.17 | 5.59 | 0.29 | May 4, 2016 | Alive | 53 |
| 10 | 1.00 | 0.09 | 1.00 | 0.04 | Aug. 30, 2011 | Exitus | 7 |
| 11 | 0.99 | 5.83 | 1.91 | 0.20 | Jul. 7, 2016 | Alive | 53 |
| 12 | 0.92 | 0.75 | 2.24 | 0.81 | Mar. 18, 2016 | Alive | 52 |
| 13 | 0.77 | 1.51 | 0.70 | 0.09 | Nov. 7, 2012 | Exitus | 10 |
| 14 | 2.21 | 2.07 | 0.73 | 0.84 | Jan. 1, 2012 | NA | 4 |
| 15 | 0.73 | 0.32 | 0.76 | 0.49 | Nov. 10, 2016 | Alive | 57 |
| 16 | 0.80 | 0.31 | 1.03 | 0.78 | Aug. 6, 2013 | Exitus | 29 |
| 17 | 1.35 | 1.05 | 0.57 | 0.03 | Dec. 10, 2011 | NA | 9 |
| 18 | 1.19 | 2.86 | 0.38 | 1.52 | Apr. 23, 2014 | Exitus | 31 |
| 19 | 1.50 | 2.87 | 0.99 | 0.50 | May 27, 2014 | Exitus | 40 |
| 20 | 0.80 | 1.33 | 0.22 | 0.43 | Sep. 1, 2012 | NA | 20 |
| 21 | 1.14 | 0.65 | 1.11 | 2.57 | Mar. 25, 2013 | NA | 14 |
| 22 | 0.50 | 2.07 | 0.26 | 2.35 | Nov. 27, 2012 | NA | 18 |

Note.
ATT, Adjacent Tumor Tissue; T, Tumor; MSP; Methylation Specific PCR; CDDP; cisplatin; CBDCA: carboplatin; M, Methylated; U, Unmethylated; NA, Not Available.

TABLE 2
Characterization at methylation and expression levels of
a cohort of 22 patients with NSCLC in statistical terms.
miR-7 methylation (%)

| | Mean | Median | Minimum | Maximum | Dispersion | p-value | |
|---|---|---|---|---|---|---|---|
| Saliva samples | 0.54 | 0.69 | 0.18 | 0.88 | 0.7 | 0.0002 | (Saliva vs NT) |
| Non Tumor (NSCLC) | 12.36 | 8.83 | 4.06 | 58.5 | 54.44 | 0.2235 | (NT vs T) |
| Tumor (NSCLC) | 17.95 | 10.49 | 3.57 | 63.08 | 59.5 | 0.0002 | (Saliva vs T) |

Expression (2^{-ΔCt})

| | Mean | Median | Minimum | Maximum | Dispersion | p-value | |
|---|---|---|---|---|---|---|---|
| | miR-7 | | | | | | |
| Non Tumor (NSCLC) | 1.13 | 1.03 | 0.5 | 2.33 | 1.83 | — | |
| Tumor (NSCLC) | 1.43 | 1.11 | 0.09 | 5.83 | 5.74 | 0.3407 | (NT vs T) |
| | MAFG | | | | | | |
| Non Tumor (NSCLC) | 0.99 | 0.68 | 0.22 | 5.59 | 5.38 | — | |
| Tumor (NSCLC) | 0.87 | 0.46 | 0.03 | 4.37 | 4.34 | 0.7146 | (NT vs T) |

Note:
p < 0.01 was considered as statistically significant changes between the groups compared (Student's t-test).

ROS Production may be in Part, Responsible of the Cisplatin Response in Cancer Cells through MAFG Overexpression

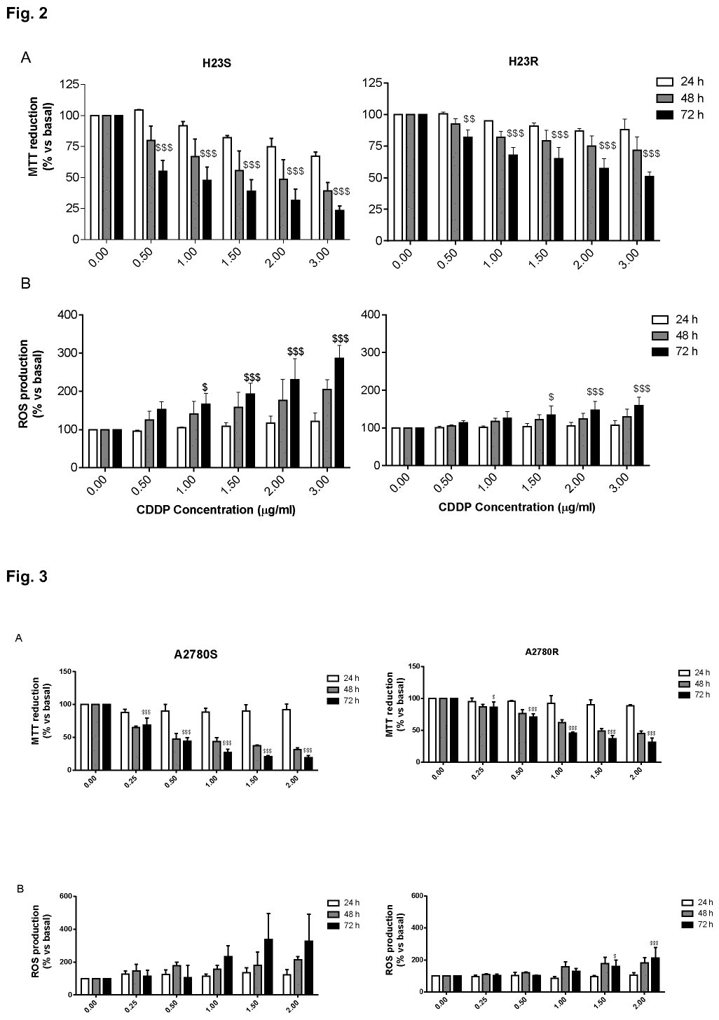

We have for the first time discovered that MAFG induces a significant increase in CDDP resistance after its overexpression in lung and ovarian cancer sensitive cells by the direct regulation of miRNA-7. MAFG is a transcription factor involved in the detoxification of reactive oxygen species (ROS); thus, we decided to analyze the ROS production in H23S/R cells after CDDP exposure. We observed that ROS are increased in sensitive cells after treatment with the drug, whereas no changes were observed in the resistant ones (FIG. 2). The differences in ROS production between sensitive and resistant cells was also confirmed in A2780 ovarian cancer cells (FIG. 3)

### MAFG Aptamers can Revert Cisplatin-Resistance by Increasing ROS Production in Cancer Cells

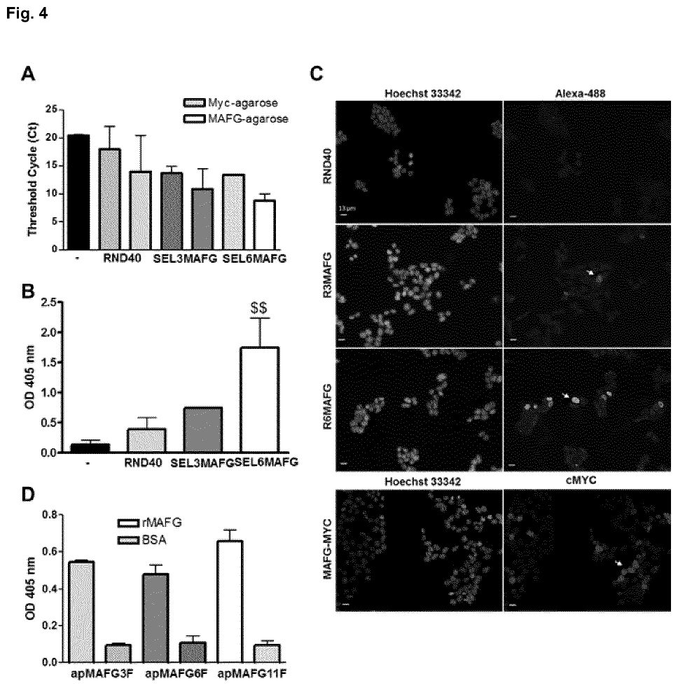

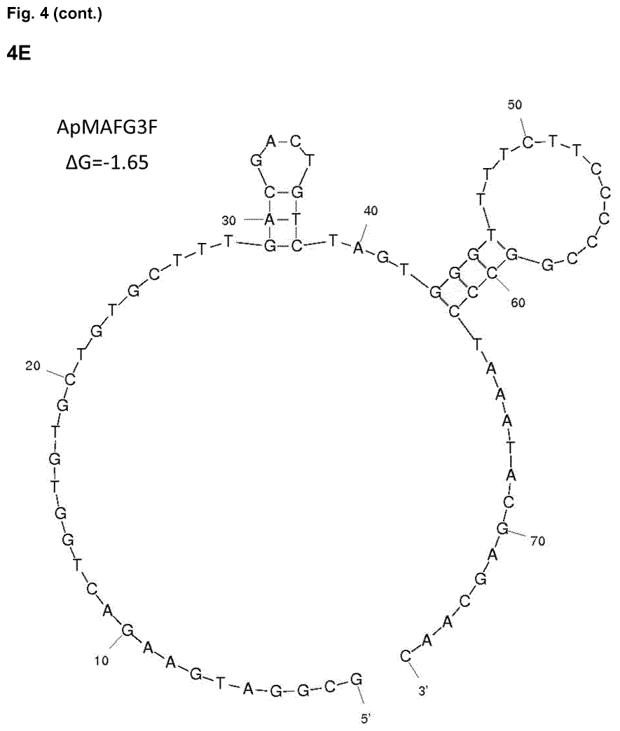

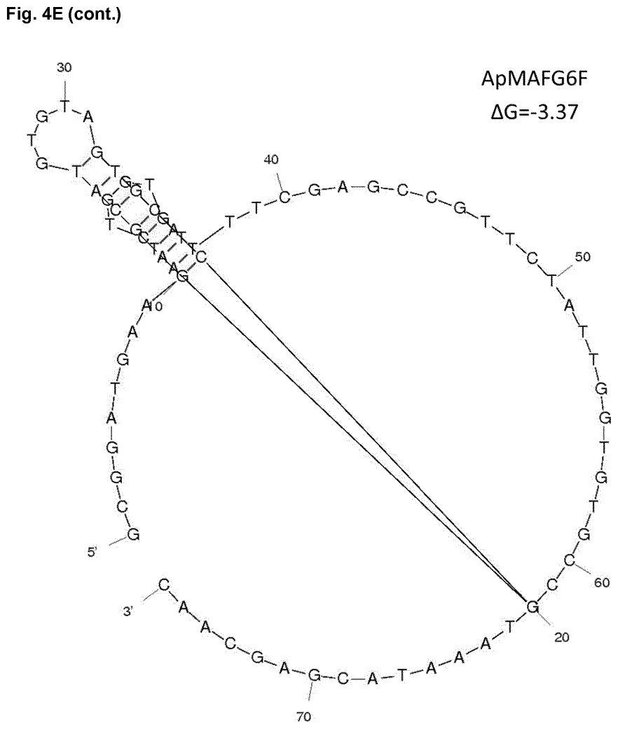

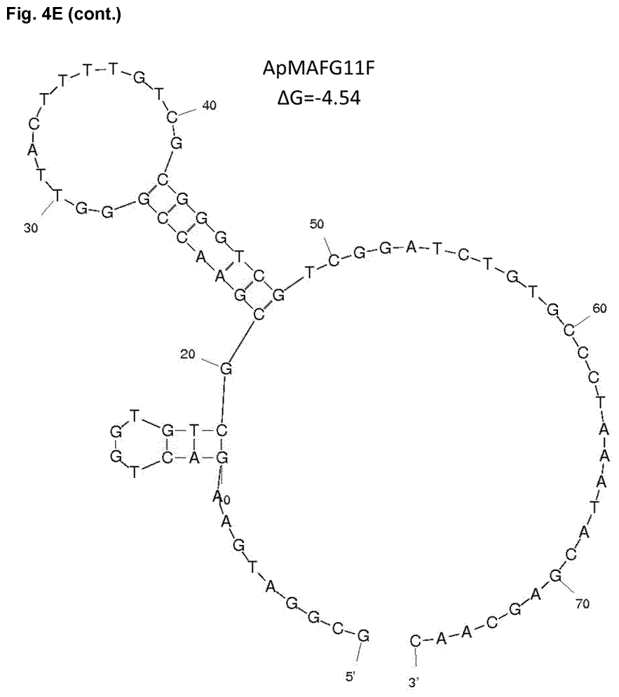

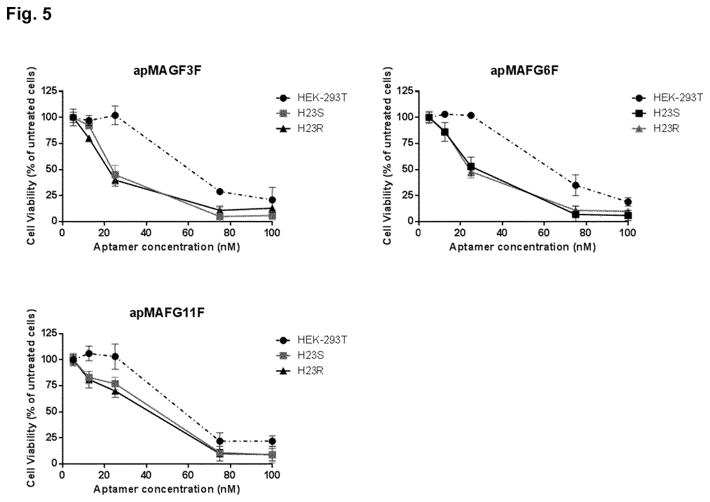

Aptamers were selected from libraries of oligonucleotides by iterative cycles of selection (SELEX methodology). We performed a selection of specific DNA aptamers against the MAFG protein identifying 3 aptamers (apMAFG3F, apMAFG6F and apMAFG11F) with high affinity against MAFG (FIG. 4). The involvement of these aptamers was tested in the normal cell line HEK293T and the sensitive and resistant phenotypes of H23. MAFG-Aptamers transfection induced the similar mortality curves in all cell lines at different doses (FIG. 5). It is important to note that the use of these aptamers did not induced mortality rates in the normal cell lines.

TABLE 3
(SEQ ID NO 3(apMAFG3F), 1(apMAFG6F) and
4(apMAFG11F))

| Name | Sequence |
|---|---|
| apMAFG3F | GCGGATGAAGACTGGTGTGCTGTGCTTTGACGACTGTC |
| | TAGTGGGTTTTCTTCCCCCGGCCCTAAATACGAGCAAC |
| apMAFG6F | GCGGATGAAGACTGGTGTCGATGCATGTGTAGTGCATT |
| | TCGAGCCGTTCTATTGGTGTGCCCTAAATACGAGCAAC |
| apMAFG11F | GCGGATGAAGACTGGTGTCGCGAACCGGGTTACTTTTG |
| | TCGCGGGTCGTCGGATCTGTGCCCTAAATACGAGCAAC |

We next tested the specificity of the aptamers for rescuing the sensitivity to CDDP in the H23. We observed that aptamer apMAFG6F decreased the CDDP-resistance at 25 nM (FIG. 6).

To finally confirm that the aptamers restore CDDP-sensitivity in H23R cells by acting over MAFG we measured the ROS production after aptamer transfection and CDDP treatment, which showed an increase of ROS production together with a decrease of cell viability of the resistant cells carrying the aptamer apMAFG6F (FIG. 7).

MAFG is Associated with Poor Prognosis in NSCLC

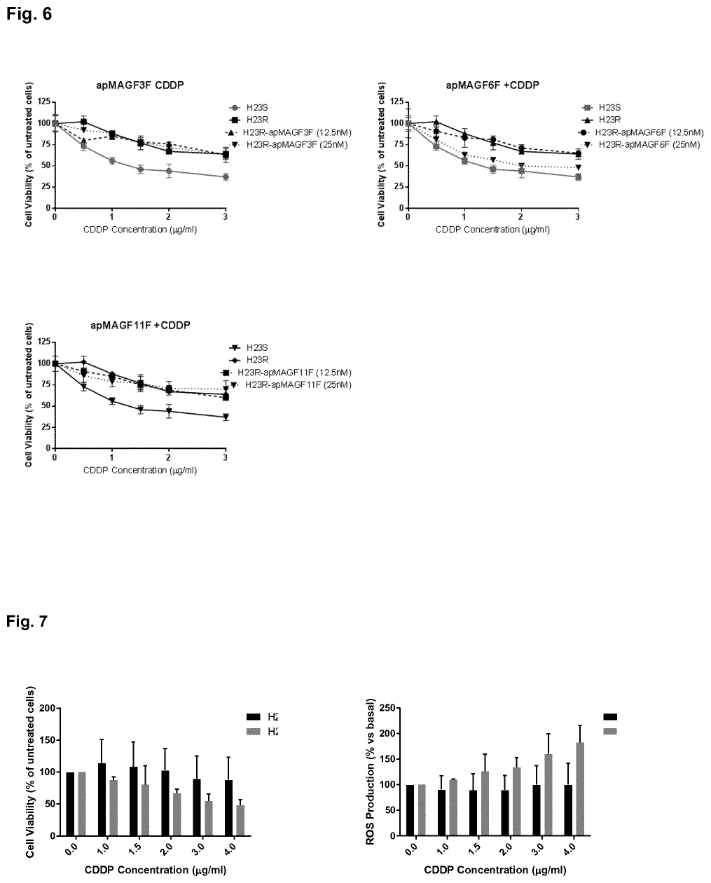

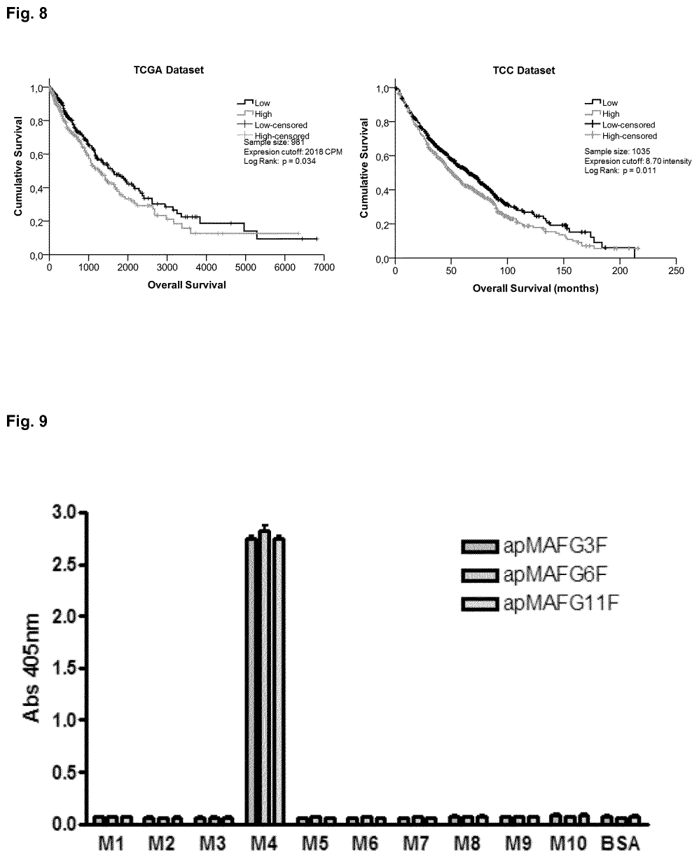

To investigate if MAFG could be useful as biomarker, we interrogated the expression levels of MAFG in a total of 981 NSCLC patients from the TOGA database and the Moffitt Cancer Center Total Cancer Care Biorepository, which included 1035 patients with NSCLC. These results showed a significant tendency towards more overall survival when MAFG is downregulated in NSCLC, with p-values of 0.034 and 0.011 for both cohorts, respectively (FIG. 8).

### Example 2

### DNA Methylation of miR-7 is a Mechanism involved in Platinum Response through MAFG Overexpression in Cancer Cells

### Materials and Methods

### Cell Culture, Treatments and Viability to CDDP

Fifteen human cancer cell lines were purchased from ATCC (Manassas, Va.) or ECACC (Sigma-Aldrich, Spain) and cultured as recommended. The CDDP-resistant variants A2780R and OVCAR3R were selected after a final exposure to 0.5 and 0.05 pg/ml cisplatin, respectively (Farma Ferrer, Spain), as previously described for H23R and H460R variants The additional 11 human cell lines, PC-3, LNCAP, H727, HT29, A549, BT474, LoVo, IMIM-PC-2, SKOV3, SW780 and IMR90, were used for further validations. For viability assays cells were treated with increasing doses of CDDP. The epigenetic reactivation drugs 5Aza-2deoxycytidine (5Aza-dC) and trichostatin A (TSA) (Sigma-Aldrich, Spain) were used at 5pM and at 300nM respectively.

### Clinical Sample and Data Collection

Formalin-Fixed Paraffin embedded (FFPE) and fresh-frozen ovarian cancer samples were collected from untreated patients and associated clinical data were obtained from Hospital Parc de Salut Mar (83 patients) and Biobank of IDIS-CHUS-HULP (55 patients) representing the most frequent ovarian cancer subtypes; all the patients underwent chemotherapy treatment after sample collection. Seven patients were also selected from stage III/IV patients from Hospital Madrid Clara Campal with a platinum treatment response classified as refractory or resistant. In addition, 22 high-grade serous carcinoma (HGSOC), were obtained from the National Cancer Research Centre (CNIO) biobank in collaboration with Dr. J. Benitez. We also collected 10 normal ovarian samples from patients who had undergone a sex reassignment surgery or tubal ligation and 10 peripheral blood mononuclear cells (PBMCs) to discard genomic imprinting. Follow-up was conducted according to the criteria of the medical oncology divisions from each institution. All the samples were processed following the standard operating procedures with the appropriate approval of the Human Research Ethics Committees at each contributing center, including informed consent within the context of research. Clinical, pathological and therapeutic data were recorded by an independent observer, and a blind statistical analysis was performed on these data.

RNA Extraction and miRNA/mRNA Array Preprocessing

RNA extraction, assessment of quality and hybridization into Agilent platforms for microRNA and mRNA microarrays and data normalization is deeply described in Supplementary Materials and Methods. The criteria used for filtering the miRNA/mRNA data were according to the packages recommended by Agilent, and were analyzed by two independent bioinformaticians. miRNA/mRNA experiments had an average expression over the 20th percentile of all average expressions and changed across the different conditions (i.e. with a coefficient of variation [CV]>5% across all samples). Global data were combined to identify those miRNAs, with inhibited expression after cisplatin treatment that were re-expressed after epigenetic reactivation, together with those genes that have in silico mRNA complementary sequences and opposite expression. For the inverse expression profiles, only those pairs (miRNA, gene) with a negative Spearman correlation coefficient and a p-value for this correlation <0.1 were considered as potential targets. The databases GeneCard (http://www.genecards.org) miRBase (www.mirbase.org), mirwalk (www.umm.uni-heidelberg.de/apps/zmf/mirwalk) and Web gestalt (www.bioinfo.vanderbilt.edu/webgestalt) were used for bioinformatics analysis. (GEO reference: GSE84201).

### RNA Isolation and Quantitative RT-PCR

Total RNA was retrotranscribed and quantitative RT-PCR analysis were performed. Samples were analyzed in triplicate using the HT7900Real-Time PCR system (Applied Biosystems, USA), and relative expression levels were calculated according to the comparative threshold cycle method (2^{-ΔΔCt}) using RNU48 or RNU6B as an endogenous control miRNAs and GADPH or β-actin as an endogenous control genes. Primers and probes for expression analysis were purchased from Applied Biosystems. Probes for gene expression are as follows: MAFG: Hs 01034678_g1; ELK1: Hs 00901847_m1; MAPKAP1: Hs 01118091_m1; ABCA1: Hs 01059118_m1; GADPH: Hs03929097_g1; 8-actin: Hs99999903_m1. Data are presented as the “change of expression in number of times” (Log10-RQ) and the error bars are expressed as the maximum estimate (RQmax) and the minimum estimate (RQmin) expression levels, representing the standard deviation of the average expression level RQ. miRNAs from human HEK-293T cell line were isolated using the miRNeasy kit (Quiagen, USA) and miR-7 expression analysis was carried out as described before, using RNU48 as endogenous control and the experimental groups transfected with 3′-UTR plasmid control and miR-NC as calibrators.

### Site-Directed Mutagenesis Assay

The full length MAFG-3′-UTR sequence (NM_002359.3 OriGene, USA) was used as a template to generate the mutants MAFG 3″UTR. Two different regions were identified by more than six bioinformatical algorithms as seed region of miR-7 binding site. Seven nucleotides within each seed region were mutated. Site-directed mutagenesis was carried out with QuikChange lightining site-directed mutagenesis kit (Stratagene, USA) according to the manufacter's instructions. The presence of both mutated seed regions and the integrity of the remaining MAFG 3UTR sequence of all constructs were validated by Sanger sequencing. The primers designed to introduce mutations were for Region2:

Fw-5'-caagtaaaccatgatatatagtgctacttccaccttaac
tttgcc-3';
Rv-5'-ggcaaagttaaggtggaagtagcactatatatcatggtt
tacttg-3';

and for Region8:

Fw-5'-ggccaagcgttccctggccagtgctatctggcctcagct
ttgttc-3',
Rv-5'-gaacaaagctgaggccagatagcactggccagggaacgc
ttggcc-3'.

### Cell Transfection and Lentiviral Transduction.

miR-7 overexpression and silencing: Cell lines were seeded at 500,000 cells/p60 plate, then transfected with 40 or 50 nM of miR-7 precursor, anti-miR-7 or negative controls (AM17100, AM 17110, AM10047 and AM17010 Ambion, USA) and using Lipofectamine 2000 (Invitrogen, USA) according to the manufacturer's protocol.

Luciferase assay: HEK-293T cells were transfected with MAFG-3′-UTR, MAFG-3′-mutated-UTR, ABCA1-3′-UTR or ELK-1-3′-UTR plasmids (OriGene, USA), and PremiR-hsa-miR-7 or Negative Control as described above. Luminiscence was assayed 24 hours later using the Kit Renilla Luciferase Assay System (Promega, USA), according to the manufacturer's instructions. Results were normalized to the Renilla luminescence from the same vector and shown as the ratio between the various treatments and cells transfected with control vector.

cDNA plasmids transfection: a Myc-DDK-tagged ORF clone of MAFG, ELK-1 or ABCA1 and the negative control pCMV6 were used for in transient transfection (OriGene, USA). H23 and A2780 cells were plated onto 60-mm dishes at 6×105 cells/dish and transfected with a negative control, MAFG, ELK-1 or ABCA1 vectors (IDs: RC221486; RC208921 and RC221861) using jet-PEI DNA Transfection Reagent (PolyPlus Transfection, USA). For stable overexpression, lentiviruses carrying ELK-1 cDNA (Applied Biological Materials, Canada) were obtained by cotransfecting 15 μg of the specific lentiviral vector (pGIPZ-nonsilencing or pLenti-GIII-CMV-hELK-1-GFP-2A-Puro) and 5 pg of each packaging vector (pCD-NL-BH and pMD2-VSV-G) in 10 million HEK 293T cells using Lipofectamine 2000 (Invitrogen, USA). Supernatants were taken at 48 hours posttransfection. A2780S cells were plated onto 60-mm dishes at 1×105 cells/dish and transduced with supernatant carrying nonsilencing or ELK-1 lentivirus, and polybrene was added (5 μg/ml).

Transfection efficacy was measured by qRT-PCR, using the sensitive cell line transfected with the negative control as a calibrator. Two independent experiments were performed in quadruplicate.

Epigenetic Validation: CpG island Identification, DNA Extraction, Bisulfite Modification, Bisulfite Sequencing and Methylation-Specific PCR

The occurrence of CpG islands (CGIs) encompassing microRNA genes or being located nearby as well as the identification of repetitive elements were assessed using various programs for CGI-revealing, listed and described in Supplementary Material and Methods. The possible gene in which the miRNA was encoded was also analyzed, searching for the presence of 5′ CGIs located in the transcriptional site. The DNA from a total of 151 samples, including tumors, controls and cultured cell was isolated, bisulfite modified and used for BS. For BS, we prefer direct sequencing, to subcloning of a mixed population of alleles to avoid potential cloning efficiency bias and artifact.

### Western Blot Analysis

Cell lines were cultured at a density of 600,000 cells per 60-mm plate, shifted into medium containing 10% fetal bovine serum for 24 h and 72 h. Twenty micrograms (20 μg) of whole-cell extracts were subjected to Western blot. The primary antibodies employed were the c-Myc-A14 (Santa Cruz, USA) and 13-tubulin (Sigma, Spain) antibodies.

### Statistical Analysis

For the identification of differentially expressed miRNAs and genes from the microarray data, we used linear models as implemented in the Limma Bioconductor package. The fixed effects were the origin of the tissue (lung/ovarian), the cell line (H460, H23, OVCAR3, A2780) and the condition (sensitive, resistant, resistant treated). The replicate is the random effect. To identify the downregulated miRNAs in resistant cells and their opposite expressed target genes, we performed the following contrasts for all the tissues (lung and ovarian) or for each tissue origin (lung or ovarian): resistant vs. sensitive and resistant-treated vs. resistant.

We then selected the candidates that fulfill the following conditions in at least 2 of the 4 cell lines interrogated: Log2(R/S)<0 AND Log2 (RT/R)>0; RvsS or RTvsR statistically different p<0.05. As a statistical method we used the unpaired T-test algorithm with Benjamini Hochberg (BH) as the FDR correction method for multiple testing corrections with statistical significance of p<0.1 in the miRNA approach and p<0.05 in the gene approach as an adjusted p-value.

Patient's clinical characteristics were described for the complete series with mean and standard deviation values or relative frequencies. The data were stratified for patients carrying methylated or unmethylated DNA, and their distributions compared with the chi-squared test or Fisher's exact test for qualitative variables, and Student's t test or the Wilcoxon-Mann-Whitney test (non-normal distribution) for quantitative variables. Overall survival and Progression free survival (PFS) were estimated according to the Kaplan-Meier method and compared between groups by means of the log-rank test. All the p-values were two-sided, and the type I error was set at 5 percent. Statistical analyses were performed using Stata 10 software.

### Results

### Establishment of Ovarian Human Cancer Cell Lines Resistant to CDDP

We have established 2 ovarian cancer cell lines resistant to CDDP, A2780-R and OVCAR3-R, that showed approximately three times more drug resistance than the paired parental cell line A2780 and OVCAR3 (3.00 and 2.96 Resistant-Index (RI); p<0.001) and a similar CDDP RI to H23R and H460R NSCLC cancer cells, (3.35 and 2.50 respectively; p<0.001).

Identification of Candidate miRNAs

As a first step to identify candidate miRNAs under epigenetic regulation and involved in the CDDP response, we searched for miRNAs showing a decrease of the expression in R versus S cells and a recovered expression after epigenetic reactivation-treated (RT) versus R cells. First, 87 miRNAs identified on the expression arrays showed a significant expression change (p<0.05) in at least one of the following conditions: R<S or RT>R; while 28 changed their expression with a p-value adj<0.1 simultaneously in both situations. By analyzing the concurrence of CGIs with the characteristics described by Takai and Jones, candidates were reduced to 10 encompassing microRNA genes or being located nearby (less than 2000 bp 5′-upstream), together with the analysis of the presence of CGIs in the gene promoter region in which the miRNA is encoded. After a pair-base-complementarity analysis in silico between miRNA and the candidate target genes that showed an opposite expression profiles; we made a functional web-based enrichment analysis with the selected genes by GOTM. This approach identified 7 miRNAs which potential target genes were involved in tumor progression: miR-7, miR-132, miR-335, miR-148a, miR-10a, miR-124 and miR-9. Mature miR-7 is generated from three different miRNA precursors in the human genome, miR-7-1, miR-7-2, and miR-7-3; we assumed expression changes were tightly associated to miR-7-3 (hereafter called miR-7) as no changes were identified on miR-7-1 and miR-7-2 probes represented in the array and it is the only precursor that presents two CGIs surrounding its genome location. We also found that some of the miRNAs showing the strongest upregulation were located at the C19MC cluster, previously linked with carcinogenesis. It presents a CGI located about 17kb from the first miRNA that was included to analyze its potential epigenetic regulation in drug resistance,

miRNA-7 as Potential Chemoresistance Candidate under Epigenetic Regulation

Firstly, we validated the expression profile of the 3 experimental conditions (S, R and RT) by qRT-PCR assay confirming the results from the microarray analysis for all 7 miRNAs in at least one of the four cell lines analyzed. Three of the seven miRNAs showed changes in their expression according to the microarray data in A2780 cells: miR-7, miR-132 and miR-10a, whereas no significant changes in expression between S and R cells were found for miR-124. The cell line OVCAR3 showed changes in miR-132 and -124, according to the array data. Although both increased expression in RT, no differences between S and R were reported. For the lung cancer model, 6 miRNAs in H23 cells and 2 miRNAs in H460 cells miRNAs were fully validated.

miR-132, miR-148a miR-335, and miR-7, were validated in at least 2 cell lines and further selected for epigenetic validation by bisulfite sequencing (BS), together with the C19MC cluster's CGI. This cluster is on the long arm of chromosome 19 and has a CGI of 2255 bp from which we analyzed 394 bp that comprises the area with the highest density of CG positions in H23, H460, A2780 and OVCAR3 cell lines. We also tested DNA from normal tissues from lung (LC), ovary (OC) and PMBCs to discard imprinting. All analyzed CpG positions were densely methylated, confirming a possible role in embryonic development, but excluding a relation between acquisition of DNA hypermethylation and drug-response. Referred to miR-132, the area analyzed was 866 bp in length, at a CGI comprising −1847/+667bp at the short arm of chromosome 17. miR-148a is located on the short arm of chromosome 7, with a nearby CGI of 1663, located 137 bp upstream from the miRNA. A 560 bp area of the CGI was analyzed. No methylation was found for both miRNAs either on the tumor cell lines or controls samples analyzed. miR-335 is located on the long arm of chromosome 7, on the second intron of the MEST002 gene transcript. A 1123 bp CGI is located in the promoter region of this transcript. We analyzed a fragment of 528 bp initially in the H23S/R, H460S/R cells and LC. The results showed methylation only in H460S/R subtypes. We extended the analysis to the additional cell lines LoVo, OVCAR3 and PC-3, and control samples, and no methylation was found in any of them.

miR-7 is located on the short arm of chromosome 19, with 2 potential regulatory CGIs: one located 861 bp before the first nucleotide of the miRNA sequence with a length of 667 bp; the second has an extension of 269 bp and comprises the miRNA sequence. Two overlapping pairs of primers were used to analyze the first CGI, covering 776 bp, which included the entire CGI and adjacent areas. The analysis was performed on the ovarian cancer cells A2780S and A2780R. We found the presence of methylation specifically in the resistant cells. The specific aberrant methylation of miR-7 in resistance was confirmed in H23R cells as well as in the cisplatin resistant cell lines IMIM-PC2 and LoVo, which present an IC₅₀ over 2 μg/ml CDDP. OC and LC were used as controls as well as nine additional tumor cell lines. In fact, the sensitive subtypes and controls presented an absence of methylation. This methylation pattern was used to design the MSP primers for the analysis of FFPE primary tumors. The second CGI, was fully methylated for all the samples tested. Therefore, the upstream CpG island of miRNA-7 was selected for our translational approach as it was the candidate downregulated through DNA methylation in CDDP-resistant cells.

miR-7 Methylation is a Potential Predictive Biomarker for Recurrence and Overall Survival in Patients with Ovarian Cancer Treated with Platinum

Response rates, overall survival or progression free survival are recommended by ASCO and ESMO Clinical Practice Guidelines Committees to assess the clinical benefit of chemotherapy treatment. Ovarian Cancer Consensus Meeting, defines ‘platinum-refractory’ as patients progressing during therapy or within 4 weeks after the last dose; ‘platinum-resistant’ patients progressing within 6 months of platinum-based therapy; ‘partially platinum-sensitive, patients progressing between 6 and 12 months; and ‘platinum-sensitive’ patients progressing with an interval of more than 12 months (GCIG Consensus). Following the international guidelines, we compared the miR-7 methylation levels with OS and PFS clinical parameters on two cohorts of 83 and 55 ovarian cancer patients all of them treated with platinum. We studied the OS for all patients and the PFS in those patients that had recurred at the end of the study to analyze the relationship between platinum response and miR-7 methylation.

We observed a 29% of methylation (24 out of 83 samples) in the cohort from Hospital del Mar, which increased to 36% (20 out of 55) in the CHUS-HULP biobank samples, a cohort enriched in serous resistant tumors. We also observed a higher percentage of methylation in HGSOC samples from and additional cohort of patients from the CNIO (50% methylated samples) and in a small group of the resistant/refractory samples from H. Madrid (57%). We also tested 10 ovarian control samples a non-tumor cell line (IMR90) and 10 PBMCs to discard imprinting and none of them were methylated (100% specificity). When correlating our results with the patient's clinical histories we obtained significant data correlating methylation and cisplatin response in the group of 33 patients that recurred. Kaplan-Meier curves show that patients relapsing before 10 months, carried preferentially methylated miR-7 tumors (80% methylated versus 14% unmethylated) (p=0.004). No differences were found in CDDP-refractory and resistant patients. Moreover, after 3 years of follow up over the 83 patients cohort, the overall survival was significantly higher in the group of patients with an unmethylated tumor in comparison with those with a methylated one (67% vs 35%, p=0.004). Similar results showing a tendency in terms of PFS and OS were also observed in the CHUS-HULP biobank cohort, although these last results were not statically significant mainly because of a size-limitation. Finally, we observed a decrease in the number of patients with higher ECOG status when the promoter region of miR-7 was unmethylated in the higher cohort of patients (p=0.025). Accordingly, 62.5% of the patients who harbored the methylated promoter presented ascites compared with 80% of the patients who did not develop ascites harboring an unmethylated promoter region (p=0.025). Those results indicate that patients carrying an unmethylated sample tended to have less aggressive tumors, with better progression free survival after platinum treatment and overall survival rates than those who carried the methylated DNA.

MAFG is a Direct Target Gene of miRNA-7

To analyze if the methylation of miR-7 is affecting the cisplatin-cell viability through the silencing of its expression, we overexpressed miR-7 in the resistant subtypes at a final concentration of 40nM. No effect on drug sensitivity was observed although efficiency of the transfection was validated by qRT-PCR, confirming the miR-7 overexpression after 72 h in both cell lines. The overexpression of higher concentration of pre-miR-7 (50nM) resulted in a decrease in cell viability, reaching levels of 63% and 52%, respectively, compared with their parental sensitive and resistant cell lines, transfected with the mimic negative control, making unfeasible to evaluate the response to CDDP, given no representative cell population was left from the cell culture after the miR-7 precursor overexpression. Thus, to fully understand the implication of miR-7 in the development of resistance, we investigated the role of the target candidate genes that showed a significant opposite expression to miR-7 and in silico complementarity. Out of the 1021 genes that accomplished both conditions we selected only those that were present in A2780 and H23 cell lines and which expression increased in R compared to S and RT subtypes, with a p-value<0.05 adjusted by FDR correction. Further functional web-based annotation using the Gene Ontology Tree Machine (GOTM) tool, grouped 149 genes in 20 significant functional groups, from which we selected MAFG, MAPKAPI, ELK-1 and ABCA1 genes because of their implication in biological functions related to tumor progression. The changes on the expression were confirmed by qRT-PCR in A2780 cells for MAFG and slighter but following the expected expression pattern for ELK-1 and in H23 cells for MAFG and ABCA1. To probe whether MAFG, ELK-1 and/or ABCA1 are target genes of miR-7, we overexpressed a precursor of miR-7 in the resistant subtypes to assess the changes in expression of the candidate target genes by qRT-PCR. As expected, the overexpression of miR-7 in A2780R resulted in a decrease of the expression of MAFG and ELK-1, compared with the resistant cell line transfected with the negative control. MAFG regulation was also confirmed in H23R cells, in which the miR-7 precursor lead also to the decrease of the potential candidate gene ABCA1. Efficiency of the miR-7 overexpresion was validated by qRT-PCR. Next, we cotransfected in HEK-293T cells the pre-miRNA-7 together with a luciferase reporter vector that carries the 3′-UTR region of each candidate gene. The cotransfection with the 3′-UTR region of MAFG, induced a reduction of the luciferase activity at both concentrations, 15 and 30 nM of the precursor, effect that was not observed when cotransfecting 3′-UTR regions of ELK-1 and ABCA1. Simultaneously, we confirmed through qRT-PCR that the pre-miR-7 was successfully transfected in the 293T cell line, for every experimental group. To fully confirm that MAFG is a target gene of miR-7, we performed directed-site mutagenesis at the predicted binding sites of miR-7 in the 3′ UTR of MAFG, at two different regions, followed by luciferase reporter assays. The significant decrease of luciferase activity observed when using the WT 3′UTR of MAFG, disappeared when we cotransfected pre-miR-7 with both constructs containing the mutated regions. Moreover, to ultimately confirm this regulation, we silenced the expression of miR-7 in A2780S that resulted in increased levels of MAFG. A2780 cells express miR-7 at a low level, which explains the low efficiency decreasing the miR-7 levels at 48h, although it was sufficient enough to observe a strong change over MAFG expression.

### The Response to Cisplatin is Mediated by MAFG Expression in Human Cancer Cell Lines

To determine if the expression of the miR-7 candidate target genes was linked to CDDP response, we conducted their in transient overexpression in the sensitive cells comparing their response to CDDP with their parental resistant and sensitive cell lines, both transfected with an empty vector.

MAFG overexpression resulted in an increase in the resistance to CDDP in A2780S cells compared with the sensitive cell line transfected with the empty vector, showing a resistance index of 1.6 (p<0.001). The same effect was also confirmed in the sensitive cell line H23S reaching a similar CDDP-RI of 1.7 (p=0.01). The overexpression of ABCA1 in H23S led to a RI of 1.5 compared with the sensitive cell line transfected with the empty vector, although it was not statistically significant (p=0.796). ELK-1 overexpression in A2780S did not change the response to CDDP after 48 h of exposure to the drug.

In order to confirm the efficiency of the transfection, we analyzed the mRNA and protein levels by qRT-PCR and western blot of the overexpressed genes. Results confirmed ectopic overexpression of MAFG, ELK-1 and ABCA1 at 72 h in both cell lines (H23S-MAFG, A2780S-MAFG, H23S-ABCA1 and A2780S-ELK-1) with an increase of 0.2, 7, 6416 and 28-folds respectively, compared with the sensitive cell lines transfected with the control vector. No changes at protein level were found between 24 and 72 hours when analyzing MAFG and ABCA1 overexpression. However, we observed a slightly protein levels of ELK1 at 24h that was not maintained at 72 h. Therefore, we performed the stable overexpression of ELK-1 by transduction assays with a lentiviral vector and compare the response to CDDP with the parental-sensitive and resistant subtypes harboring a nonsilencing vector (A2780S/R -NS). As previously observed in the “in transient” experimental assays, ELK-1 overexpression did not change the sensitivity to CDDP; however, it induced an strongly increase in the number of cells at 0 μg/ml dose, that allowed to maintain higher ratios of survival fraction when treated with CDDP, compared with the control sensitive cell line. We also confirmed the success of the overexpression by q RT-PCR.

### Example 3

### Materials and Methods

### Cell Cultures and Treatments

The human cancer cells H23 and A2780, and the HEK-293T epithelial cells were purchased from ATCC (Manassas, USA) and ECACC (Sigma, Spain) and cultured as recommended. The CDDP-resistant variants H23R and A2780R were established in our laboratory as previously described [13, 17], using CDDP (Farma Ferrer, Spain) for cell viability assays. To validate the results obtained from the resistant cell lines established in our laboratory, we also used the CDDP-resistant lung cancer cell line H1299, with IC50 over 7, purchased from ATCC and maintained as recommended.

### Clinical Sample and Data Collection

Fresh frozen T and ATT paired samples were obtained from 22 patients with NSCLC from La Paz University Hospital. All patients had both a perioperative PET-CT scan showing localized disease and a pathological confirmation of stages after having undergone a complete resection for a histologically confirmed early NSCLC. Follow-up was conducted according to the criteria of the medical oncology division from the institution. In addition, 5 saliva samples from healthy donors were used as controls. All samples were collected after the approval of the appropriate Human Research Ethics Committee at the contributing center, including an informed consent within the context of research. Clinical, pathological and therapeutic data were recorded by an independent observer, and a blind statistical analysis was performed on the data.

### RNA Isolation, RT-PCR and Quantitative RT-PCR

Total RNA from surgical samples was isolated, reverse transcribed and quantitative RT-PCR analysis was performed as previously described [17, 18]. Samples were analyzed in triplicate using the HT7900Real-Time PCR system (Applied Biosystems, USA). Relative expression levels were calculated according to the comparative threshold cycle method (2-ΔΔCt) using GAPDH as an endogenous control gene and RNU48 as endogenous control miRNA. Primers and probes for expression analysis were purchased from Applied Biosystems (MAFG: Hs 01034678_g1; GAPDH: Hs03929097_g1; miRNA-7: 000268; and RNU48: 001006).

For semiquantitative RT-PCR, 500 ng of total RNA isolated from cell lines was used for RT reaction using PrimeScript™ RT Master (Clontech-Takara, USA) and subsequent semiquantitative PCR using Promega PCR Mix (Promega, USA) as previously described [19]. Relative quantification was performed by measuring the intensity of band amplified using ImageJ software. Primers for HMOX1 (F: 5′-TGAGTTTCAAGTATCCTTGTTGACAC-3′; R: 5′-CTTGGTCTAACTTTTGTGTGAAATAA-3′), MAFG (F: 5′-TCAGATTTCAGAGGAATACCCAGCAG-3′; R: 5′-TGATCACCAGTCAGAAGTGTACACAC-3′) and GAPDH (F: 5′-GAGAGACCCTCACTGCTG-3′; R: 5′-GATGGTACATGACAAGGTGC-3′) were designed to include the probe from the array in order to assure the correct transcript identification.

### DNA Extraction, Bisulfite Modification and Quantitative Methylation-Specific PCR

DNA from a total of 44 surgical samples from patients with NSCLC was isolated and bisulfite modified as previously described [18] and was used to analyze miRNA-7 methylation status. For quantitative methylation-specific PCR (qMSP), we used the primer/probe set to detect levels of either methylation (F: 5′-GGGTGGGGTTTTTTAAGAATC-3′; R: 5′-ACATTCTCCTCCTTCGATCG-3′; Probe: 5′-FAM-ACCCCTCTTCGTTCTCGAT-3′) or unmethylation (F: 5′-GGGGTGGGGTTTTTTAAGAATT-3′; R: 5′-ATAACATTCTCCTCCTTCAATCA-3′; Probe: 5′-VIC-ACCCCTCTTCATTCTCAAT-3′). All assays were performed in duplicate using the QuantiTect Multiplex PCR Kit (Qiagen, USA) and the HT7900 Applied Biosystems. The percentage of methylation of each sample was calculated according to previously published reports.

### Western Blot Analysis

Proteins (30 pg) from cell lysates were resolved by sodium dodecyl sulfate-polyacrylamide gel electrophoresis and transferred to Immobilon-P membranes (Millipore lberica SA, Madrid, Spain). Membranes were incubated with anti-Nrf2 at 1:10000, anti-HO-1 at 1:10000 (Abcam) and anti-β-actin at 1:100,000 (Sigma, Madrid, Spain). Peroxidase-conjugated secondary antibodies (1:10000) were used to detect proteins by enhanced chemiluminescence detected by Advance Western-blotting Detection Kit (GE Healthcare, Barcelona, Spain).

### In Silico Databases: The Cancer Genome Atlas and Total Cancer Care

The Cancer Genome Atlas (TCGA) data: We obtained RNA sequencing data for the MAFG of 984 NSCLC tumors from the TCGA. The raw reads were quantified by RSEM in order to determine the read counts for each gene and miRNA (calculated separately). Then, we filtered out genes and miRNAs having less than one count-per-million reads in all samples. The normalization process was performed with trimmed mean of M-values to obtain the MAFG sequence count data in all patients.

Total Cancer Care (TCC): We obtained MAFG gene expression data for 1035 lung cancer samples from the Moffitt Cancer Center Total Cancer Care Biorepository that were assayed on a custom Affymetrix 2.0 microarray. Normalized intensity values for MAFG probe sets were obtained and the probe with highest average intensity was retained for gene expression analysis.

### ROS Measurement

H23/A2780 cells were cultured in 96-well black plates at density of 10,000 cells/well. Cells were treated with 6 different doses of CDDP for 24, 48 and 72h. Then treatments were removed and cells were incubated with the fluorescent probe 2′,7′-dichlorodihydrofluorescein diacetate (H2DCFDA) (10 μM) for 45 minutes in RPMI medium (FBS-free). Cells were washed twice with RPMI (10% FBS), and fluorescence was recorded in a Fluostar Optima at 520 nm after excitation at 485 nm. At the end of the experiment, solutions were replaced for fresh media containing MTT (0.5 mg/ml concentration) in order to determinate viability. ROS production was calculated dividing the mean H2DCFDA fluorescence by the mean viability. Data were normalized with respect to basal conditions that were considered as 100%.

### Aptamer Selection for MAFG

Selection of DNA aptamers for MAFG was performed using the SELEX method with several modifications. First, MAFG-Myc overexpressed in HEK293T cells were bound to Myc-agarose beads (Thermo Scientific 20168), following the supplier's instructions. The same amount of lysates from HEK293T cells transfected with empty vector was immunoprecipitated in parallel and used for the contraselection step. Synthetic random ssDNA (IBA Life Sciences, Germany), containing a central randomized region of 40 nucleotides flanked by two conserved 18-nucleotide regions in each end (RND40, 5′-GCGGATGAAGACTGGTGT-40N-GCCCTAAATACGAGCAAC-3′) was denatured at 95° C. for 10 minutes and then cooled on ice for 10 minutes. Further steps of the selection were followed as previously described with minor modifications. Finally, contraselection prepared as above, was performed after round 2 and 5.

i) Analysis of aptamer-MAFG complexes by real-time PCR: The aptamer populations obtained after 3 and 6 rounds of selection (SEL3MAFG and SEL6MAFG) or RND40 were incubated at 0.4 nM with 20 pmoles of MAFG bound to Myc-agarose for 1 hour at 37° C. In parallel, the same amount of each aptamer was incubated with the same volume of Myc-agarose resin without MAFG. After centrifugation at 12,000 g for 10 minutes, complexes were washed four times with 250 μl of selection buffer and confirmation the aptamers presence was performed.

(ii) Enzyme-linked oligonucleotide assay (ELONA): Aptamers were labeled by PCR using 5′ digoxigenin-labeled F3/5′ digoxigenin-labeled R3 primers (IBA Life Sciences). To assess the enrichment of the selected population and the affinity of the individual aptamers for the target, MAFG-Myc (50 ng) was incubated with the rounds 3, 6 of aptamers or the RND40 library. OD405nm values were determined using a SpectraFluor microplate reader (TECAN, Barcelona, Spain).

(iii) Aptacytochemistry: HEK293T cells were seeded on glass coverslips pretreated with poly-L-lysine (Sigma-Aldrich). After 16-24 hours, the cells were transfected with 0.4 μg of MAFG-Myc plasmid using Lipofectamine 2000 (Invitrogen) following the manufacturer's instructions. Twenty-four hours posttransfection, the cells were fixed with cold methanol for 20 minutes at −20° C. and followed incubation with aptamer populations SEL3MAFG and SEL6MAFG. Co-localization was assessed by confocal microscopy using a Nikon ECLIPSE Ti-e inverted fluorescence microscope equipped with a Nikon Cl laser scanning confocal microscope system (Nikon, Tokyo, Japan) and a 60x oil immersion objective.

(iv) Aptamer cloning, sequencing and secondary structure prediction: The dsDNA products with “A”-overhangs from SEL6MAFG were cloned onto pGEM-T Easy-cloning vector (Promega), following the manufacturer's instructions. Individual clones were sequenced using T7 (5′-TAATACGACTCACTATAGGG-3′) and Sp6 primers (5′- ATTTAGGTGACACTATAGAA-3′) provided by the Sequencing core. Selected ssDNA molecules were subjected to secondary structure prediction using mFold software (http://mfold.rna.albany.edu/?q=mfold/DNA-Folding-Form) 41 at 37° C. in 150 mmol/l[Na+] and 1 mmol/l[Mg+2].

Aptacytochemistry and Aptahistochemistry with Selected Dig-Labeled Aptamers

Following the methodology described above, H23R cells were incubated with 6 pmol of digoxigenin-labeled aptamers (IBA Life Sciences) in selection buffer for 1 hour at room temperature. Subsequently, cells were washed three times with PBS and incubated with anti-digoxigenin conjugated with 5′ AlexaFluor 488 (R&D systems, USA) at 1/500 dilution in blocking buffer for 1 hour at room temperature, as secondary antibody. Finally, the cells were mounted on glass slides using glycerol-buffer containing p-phenylenediamine and 1/750 dilution of Dapi for nuclear staining. Controls were made by omitting the aptamer. Colocalization was assessed by confocal microcopy as described in (iii).

The formalin-fixed and paraffin-embedded tissue sections of patients with lung cancer were baked 15 minutes at 60° C. Deparaffinization was performed by two washes of xylene for 10 minutes each. Tissue sections were rehydrated by a sequential wash in 100%, 90%, 80%, 70% ethanol and distilled water for 5 minutes each. Antigen retrieval was achieved by heat treatment in a pressure cooker for 2 minutes in 10 mM citrate buffer (pH 6.5). Endogenous peroxidase activity of tissues was blocked with 0.3% H2O. The primary binding reaction was performed with 10 pmol/ml of digoxigenin-conjugated aptamers overnight at room temperature. The secondary binding reaction was performed with anti-digoxigenin conjugated with POD (Roche; 1/200 dilution in TBS) for 45 minutes. Staining was done using the Immunoperoxidase DAB kit (Master Diagnostica), according to the manufacturer's instructions. The sections were counterstained with hematoxylin.

### Cell Transfection and Viability Assays

HEK293 and H23S/R cells were seeded into 96-well plates then transfected with 5, 12.5, 25, 75 and 100 nM of MAFG aptamers or treated with PBS-Mg using Jet Pei PolyPlus transfection reagent (PolyPlus Transfection, USA). The dose of 25 nM aptamer was afterwards used in combination with increased doses of CDDP to assay viability and ROS production as described above. To confirm the aptamer-transfection efficiency cells were seeded in 24-well plates for aptamer isolation and confirmation of aptamer transfection by quantitative analysis. A Myc-DDK-tagged ORF clone of MAFG the negative control pCMV6 were used for in transient transfection (OriGene, USA) in combination with MAFG-aptamers following the same procedure.

Statistical analysis

The data were compared using the chi-squared test or Fisher's exact test for qualitative variables, and Student's t test or the Wilcoxon-Mann-Whitney test (non-normal distribution) for quantitative variables. Correlation of quantitative variables was analyzed by Pearson's test. For the in silico databases, the data were stratified for patients with high or low expression of MAFG according to the median of the gene expression (TCGA cutoff: 2018 counts per million; TCC cutoff: 8.70 of intensity probe). Overall survival was estimated according to the Kaplan-Meier method and compared between groups by means of the Log Rank test. All the p-values were two-sided, and the type I error was set at 5 percent. Statistical analyses were performed using SPSS 20 software.

### Results

MAFG Overexpression is Associated with a Poor Prognosis in Patients with Non-Small Cell Lung Cancer

As shown in example 2, the epigenetic regulation of miR-7 through DNA methylation is a mechanism involved in platinum resistance in cancer cell lines directly regulating the action of

MAFG, which is overexpressed in resistant phenotypes. We explored the involvement of the miR-7/MAFG axis in non-small cell lung cancer (NSCLC) primary tumors. We first analyzed the quantitative DNA methylation levels of miR-7 and RNA expression levels of miR-7 and MAFG in a cohort of 22 paired samples from fresh frozen tumors (T) and adjacent-tumor tissue (ATT) from patients with NSCLC and controls.

We observed an increase in the data dispersion in the NSCLC T samples versus the ATTs in miR-7 methylation and expression levels. The higher dispersion was statistically significant in both groups only when compared with saliva samples from healthy donors (p=.0002). A negative tendency in the correlation between the percentage of methylation and expression levels of miR-7 was found only in the T samples. The opposite tendency was found, toward more expression of MAFG in T samples, when the percentage of miR-7 methylation increased. Neither tendency was statistically significant, probably due to the limited sample numbers; however it is interesting to note that the two samples that showed the highest dispersion for MAFG expression presented a miR-7 methylation level over 20%.

To determine whether MAFG expression correlated with clinical outcome in patients with NSCLC, we investigated the gene expression levels in 984 patients with NSCLC from the TOGA database and 1035 patients with NSCLC from the Total Cancer Care Biorepository at the Moffitt Cancer Center. When comparing data from both data sets, we observed statistical significance according to the median level of MAFG expression; patients with low expression levels had a clearly increased overall survival compared with the group of patients with high MAFG expression levels, with p-values of 0.020 and 0.011 for both cohorts, respectively.

### MAFG Overexpression Induce CDDP Resistance, Targeting ROS

MAFG is a transcription factor involved in the detoxification of ROS, whose expression is increased in the resistant cell phenotypes H23R and A2780R, which mediates their resistance to CDDP. Thus, we explored whether MAFG influenced oxidative stress in our experimental model of paired sensitive/resistant cells by analyzing ROS production in cells after CDDP exposure.

First, we confirmed the response to CDDP at 24, 48 and 72 h after exposure to the drug for both cell lines H23S and H23R. H23R cells present a resistance index >3. ROS levels were increased in sensitive cells after CDDP treatment compared with resistant cells, reaching 300% and 159% ROS production at 3.00 μg/ml CDDP, respectively, versus basal untreated cells, p<0.001. The response to CDDP and the differences in ROS production between sensitive and resistant cells were also confirmed in A2780S/R ovarian cancer cell lines, in which we observed an effect of CDDP treatment after 48 h of exposure. Moreover, we found that the expression of ROS-detoxifying genes, such as HMOX1, NQO1, GSTO2 or GPX7, was upregulated in these resistant cell lines (p<0.05). We selected HMOX1 and NRF2 for semiquantitative real-time polymerase chain reaction (RT-PCR) expression pattern validation, because of their close relationship with MAFG action in the detoxification process. NRF2 expression did not changed after cisplatin treatment, however, the resistant cells showed higher protein levels of NRF2 and it was also observed a slight increase in HMOX1 levels. In addition, we observed a direct relationship between the expression of MAFG and the Resistance Index in all our lung cancer cell lines.

### Identification of Aptamers Binding MAFG

Aptamers were selected from libraries of oligonucleotides by iterative cycles of selection (systematic evolution of ligands by exponential enrichment [SELEX] methodology). We performed 6 rounds of selection and 2 contraselection rounds (after rounds 2 and 5), using a Myc agarose resin that binds the MAFG-Myc protein overexpressed in HEK293T cells. To assess the enrichment and the affinity of the population obtained after successive rounds of selection, we performed three types of assays: (i) analysis of aptamer-MAFG complexes by RT-PCR; (ii) enzyme-linked oligonucleotide assay (ELONA) assays and; (iii) aptacytochemistry. Quantitative PCR assays were performed using aptamers that bind to MAFG-resin complexes as a template. These assays allowed us to confirm that the selected aptamers were specific against the target and not the Myc-agarose resin. The amount of aptamers recovered in the presence of MAFG was 3 Cts lower than in that of the RND40 and SEL3MAFG population, whereas SEL6MAFG was 4.5 Cts lower in the presence of MAFG, indicating a higher amount of aptamers bound in this round. In addition, ELONA assays showed a statistically significant increase in the signal of the population obtained after round 6 relative to round 3 or to the initial RND40 population. Finally, we studied the capacity of the aptamer population of each round to recognize overexpressed MAFG in the cells by aptacytochemistry. For this purpose, HEK293T cells transiently transfected with MAFG-Myc were incubated with the initial RND40 population or with that of round 3 or round 6, labeled with Alexa-488. The results obtained by fluorescence microscopy indicated that RND40 showed a very low signal with a diffuse cytoplasmic pattern, whereas SEL3MAFG and most prominently SEL6MAFG aptamers showed a high nuclear signal in several cells. This pattern was very similar to that obtained by incubation with the Myc antibody. In view of these results, we cloned and sequenced the SEL6MAFG population and identified three sequences (apMAFG3F, apMAFG6F and apMAFG11F) with high binding capacity to the target in the nanomolar range, reaching values of four times that of the BSA protein. To predict the most stable secondary structures of the MAFG aptamers, we performed a bioinformatic analysis of their sequences using mFold software.

MAFG Aptamers can be used to Detect MAFG Levels in NSCLC Samples

We next performed aptacytochemistry on H23S and H23R cells with the individual digoxigenin-labeled aptamers apMAFG3F, apMAFG6F and apMAFG11F. We observed that individual aptamers were more present in H23R cells than in H23S, mostly in the nuclei of the cells and specifically accumulated in quantifiable dots, according to the cellular localization of MAFG. Quantification of the number of dots per nucleus showed remarkable differences between H23R and H23S for apMAFG3F and apMAFG6F, whereas no differences were observed for apMAFG11F. Interestingly, apMAFG3F was also present in the cytoplasm, whereas apMAFG6F was preferentially located at the nuclei.

We then took advantage of the aptacytochemistry to perform an aptamer-based histochemistry in 20 tumor samples from our cohort of patients with known mRNA levels of MAFG. We found that aptamers were able to identify MAFG in the nucleus of various cell types with differential staining intensity between the tumors. To determine the correlation between the aptahistochemistry and the mRNA levels of MAFG, we calculated the percentage of cells with positive staining from the total number of cells computed at four different areas randomly selected for each sample. We observed that classifying the patients according to their MAFG expression levels, those samples with higher MAFG levels presented a significant increment in the percentage of positive cells after aptahistochemistry staining with both apMAFG3F and apMAFG6F (p<0,05 and p<0,01, T-test respectively).

apMAFG6F Increases ROS Production and Restores Cell Sensitivity to CDDP

To gain insight into the biological effect of the identified aptamers and to optimize the dose for a functional analysis, we worked with the normal cell line HEK293T and the sensitive and resistant phenotypes of the lung cancer cell lines H23S/R. MAFG aptamer transfection induced similar mortality curves in the cancer cell lines, while it appeared to induce less toxicity in HEK293T, which showed better viability curves for the three aptamers tested. Concentrations higher than 25 nM induced a high mortality rate in all the cells tested, and the same effect was also observed when two intermediate doses selected between the 25 nM and 75 nM concentrations were tested in H23R. The 25 nM concentration was chosen for functional analysis because it is the minimal dose that exerts an effect on viability of tumor cells but not normal cells. The efficiency of aptamer transfection at this dose was confirmed by quantitative PCR. We next tested the specificity of the aptamers for rescuing the sensitivity to CDDP. Aptamer apMAFG6F decreased the CDDP resistance at 25 nM (resistance index 2.63 vs. 2.0), whereas no changes were observed for aptamers apMAFG3F or apMAFG11F. We confirmed these results in H1299, an additional lung cancer tumor cell line highly resistant to CDDP with an IC50 of 10 μg/ml. In fact, we verified the ability of apMAFG6F to increase CDDP sensitivity in H1299, as we observed a decrease in the IC50 to 4.5 μg/ml platinum in comparison with the untransfected cell line. This increase in sensitivity to the drug was accompanied by a decrease in MAFG and HMOX1 expression in the cells carrying apMAFG6F. In addition, we overexpressed MAFG in H23S cells and combine the overexpression with apMAFG6F, which rescued the phenotype and re-sensitized the cells. We also confirmed the success of the overexpression and aptamer transfection by qRT-PCR.

To ultimately confirm that apMAFG6F was restoring CDDP sensitivity in H23R cells by acting on the ROS, we measured ROS production after combining aptamer transfection and CDDP treatment. We observed a linear dose-response relationship between increasing amounts of CDDP and the production of ROS at 4 μg/ml of CDDP (p<0.005) in the resistant cells carrying the apMAFG6F compared with the untransfected cells. This result was also consistent with the decrease in cell viability that we also measured in parallel in this experiment. Semiquantitative RT-PCR of MAFG and HMOX1 showed a decrease in their expression in aptamer-transfected H23R cells after platinum treatment compared with H23R.

1. In vitro use of the amino acid sequence comprising RTLKNRGYAASCR or RTLKNRGYAASCRVKRVTQ, wherein the amino acid sequence constitutes a binding site for a therapeutical binding molecule selected from the group consisting of aptamers, antibodies, and antibody fragments selected from the list consisting of Fv, scFv, Fab, F(ab′)2, Fab′, scFv-Fc, diabodies, or any fragment whose half-life has been increased by chemical modification, as a target for a screening method for compounds useful in a method of treatment to decrease or inhibit chemotherapy resistance to chemotherapy regimens based on platinum.

2. The in vitro use according to claim 1, wherein aminoacids 57ARG, 60LYS, 61ASN, 62ARG, 63GLY, 64TYR, 65ALA, 66ALA, 67SER, 68CYS, 69ARG, 70VAL, 71LYS, and 75GLN of the epitope of MAFG comprising amino acids 57 to 75 RTLKNRGYAASCRVKRVTQ, are used as a target.

3. A pharmaceutical composition comprising a binding molecule capable of decreasing or inhibiting, in vitro or ex vivo, the expression and/or activity of MAFG in a cancer cell, in comparison to the expression and/or activity of MAFG in a non-cancerous cell or in comparison to a reference value, for use in a method to decrease or inhibit chemotherapy resistance to chemotherapy regimens based on platinum, wherein said binding molecule binds to an epitope or to an isolated synthetic peptide comprising the amino acid sequence RTLKNRGYAASCR or RTLKNRGYAASCRVKRVTQ.

4. The composition for use according to claim 3, wherein said binding molecule binds to an epitope or to an isolated synthetic peptide comprising the amino acid sequence IVQLKQRRRTLKNRGYAASCR or IVQLKQRRRTLKNRGYAASCRVKRVTQ.

5. The composition for use according to any of claim 3 or 4, wherein the binding molecule is a compound selected from the group consisting of aptamers, antibodies, and antibody fragments selected from the list consisting of Fv, scFv, Fab, F(ab′)2, Fab′, scFv-Fc, diabodies, or any fragment whose half-life has been increased by chemical modification.

6. The composition for use according to claim 5, wherein the binding molecule binds to aminoacids 57ARG, 60LYS, 61ASN, 62ARG, 63GLY, 64TYR, 65ALA, 66ALA, 67SER, 68CYS, 69ARG, 70VAL, 71 LYS, and 75GLN of the epitope of MAFG comprising amino acids 57 to 75 RTLKNRGYAASCRVKRVTQ (amino acids 57 to 75).

7. The composition for use according to claim 6, wherein the binding molecule is an aptamer.

8. The composition for use according to claim 7, wherein the aptamer is apMAFG6F of SEQ ID NO 1.

9. The composition for use according to anyone of claims 3 to 8, wherein the chemotherapy regimen based on platinum is a chemotherapeutic drug selected from cisplatin and/or carboplatin.

10. A binding molecule as defined in claim 3, in combination with at least one chemotherapeutic drug based on platinum for use in the treatment of cancer, for use in the prevention of cancer metastasis and/or for use in the prevention of cancer recurrence or for increasing overall survival in a subject.

11. The binding molecule in combination with at least one chemotherapeutic drug based on platinum for use according to claim 10, wherein the cancer cell expresses MAFG.

12. The binding molecule in combination with at least one chemotherapeutic drug based on platinum for use according to claim 10 or 11, wherein cancer is selected from a melanoma, a breast cancer, a thyroid cancer, a prostate cancer, a colon cancer, a rectal cancer, an oesophagus cancer, a gastric cancer, an ovarian cancer, a lung cancer, a pancreatic cancer, a glioma, an adrenocortical carcinoma, a pediatric solid malignant tumor, a leukaemia, a multiple myeloma and a sarcoma.

13. The binding molecule in combination with at least one chemotherapeutic drug based on platinum for use according to anyone of claims 10 to 12, wherein the at least one chemotherapeutic drug is selected from cisplatin and/or carboplatin or any platinum derived chemotherapeutic compound.

14. The binding molecule in combination with at least one chemotherapeutic drug based on platinum for use according to anyone of claims 10 to 13, wherein the subject is a mammal, preferably a human being.

15. The binding molecule in combination with at least one chemotherapeutic drug based on platinum according to claim 14, wherein the subject is a human being suffering of a cancer and resistant to chemotherapy based on platinum.

16. A composition comprising at least one binding molecule as defined in any of claims 2 to 9 for use in the prevention of cancer metastasis and/or for use in the prevention of cancer recurrence in a subject, in combination, simultaneously, separately or sequentially, with at least one chemotherapeutic drug based on platinum.
